(19) United States
(12) Patent Application Publication
(10) Pub. No.: US 2025/0179202 A1
(43) Pub. Date: Jun. 5, 2025

(54) Antibodies That Specifically Bind to TL1A and Methods of Treating Skin Disease

(71) Applicant: Cephalon LLC, West Chester, PA (US)

(72) Inventors: Lynn Dorothy Poulton, Macquarie Park (AU); Matthew Pollard, Pullenvale (AU); Anthony G. Doyle, Macquarie Park (AU); Bridget A. Cooksey, Macquarie Park (AU); Vanya Pande, Macquarie Park (AU); Adam W. Clarke, Macquarie Park (AU)

(73) Assignee: Cephalon LLC, West Chester, PA (US)

(21) Appl. No.: 18/939,176

(22) Filed: Nov. 6, 2024

Related U.S. Application Data
(62) Division of application No. 17/538,530, filed on Nov. 30, 2021, now Pat. No. 12,162,946.
Application No. 17/538,530 is a division of application No. 16/164,509, filed on Oct. 18, 2018, now Pat. No. 11,220,549.
Application No. 16/164,509 is a division of application No. 15/267,213, filed on Sep. 16, 2016, now Pat. No. 10,138,296.
(60) Provisional application No. 62/220,442, filed on Sep. 18, 2015.

(51) Int. Cl. C07K 16/28; A61K 39/00; A61P 11/06; A61P 37/06; C07K 16/24

(52) U.S. Cl. CPC C07K 16/2875; A61P 11/06; C07K 16/241; A61K 2039/505; A61P 37/06; C07K 2317/21; C07K 2317/33; C07K 2317/565; C07K 2317/76; C07K 2317/92

(57) ABSTRACT

Recombinantly expressed variant antibodies that have enhanced affinity for TL1A and enhanced potency relative to the parent antibody from which they were derived are provided. The antibodies inhibit the interaction between TL1A and the death receptor 3 (DR3). The antibodies, or a composition thereof, may be used to treat one or more of asthma, COPD, pulmonary fibrosis, cystic fibrosis, inflammatory bowel disease, a gastrointestinal disease associated with cystic fibrosis, Crohn's disease, colitis, ulcerative colitis, irritable bowel syndrome, eosinophilic esophagitis, atopic dermatitis, eczema, scleroderma, arthritis, or rheumatoid arthritis.

### CROSS REFERENCE TO RELATED APPLICATIONS

This application is a divisional of U.S. application Ser. No. 17/538,530, filed on Nov. 30, 2021, which is a divisional of U.S. application Ser. No. 16/164,509 filed on Oct. 18, 2018 (Issued as U.S. Pat. No. 11,220,549 on Jan. 11, 2022), which is a divisional of U.S. application Ser. No. 15/267,213 filed on Sep. 16, 2016 (Issued as U.S. Pat. No. 10,138,296 on Nov. 27, 2018), which claims the benefit of U.S. Provisional Application No. 62/220,442, filed on Sep. 18, 2015, the contents of which are incorporated by reference herein, in their entirety and for all purposes.

### REFERENCE TO A SEQUENCE LISTING

This application includes a Sequence Listing submitted electronically as an XML file named 01183-0322-03US_Sequence Listing ST26, created on Oct. 30, 2024 with a size of 108,053 bytes. The Sequence Listing is incorporated by reference herein.

### FIELD OF THE INVENTION

This disclosure relates generally to the field of antibody engineering. More specifically, this disclosure relates to variant antibodies that bind specifically to TL1A, and which inhibit the interaction between TL1A and the death receptor 3 (DR3). In some aspects, the antibodies also inhibit the interaction between TL1A and the decoy receptor 3 (DcR3). The antibodies have improved potency relative to the parent antibody from which the variants were derived.

### BACKGROUND OF THE INVENTION

Various publications, including patents, published applications, accession numbers, technical articles and scholarly articles are cited throughout the specification. Each of these cited publications is incorporated by reference, in its entirety and for all purposes, in this document.

TNF-like ligand 1A (TL1A, syn. TNF superfamily member 15 (TNFSF15); TL1 and VEGI) is a member of the tumor necrosis factor superfamily, which is expressed by antigen presenting cells (including dendritic cells, B cells and macrophages), CD4+ and CD8+ T cells and endothelial cells. TL1A can be expressed on the cell surface or secreted as a soluble cytokine. The receptor for TL1A, Death Receptor 3 (DR3) is expressed by a variety of cells, including CD4+ and CD8+ T cells, NK cells, NKT cells and FOXP3+ regulatory T (Treg) cells and type-2 and type-3 innate lymphoid cells (ILC2 and ILC3).

TL1A can also bind a decoy receptor (DcR3), which is a competitive inhibitor of DR3. DcR3 also acts as a decoy receptor for Fas-ligand (Fas-L) and lymphotoxin-like inducible protein that competes with glycoprotein D for binding herpesvirus entry mediator on T-cells (LIGHT). Accordingly, DcR3 is an important regulator of several signal transduction pathways.

The TL1A/DR3 signalling pathway has been implicated in several biological systems, which are associated with human diseases. For example, TL1A has been shown to play a role in immunity, angiogenesis, and homeostasis of barrier tissues. Inhibiting TL1A interaction with DR3 also has been shown to promote a therapeutic benefit in several immune-mediated conditions, such as experimental autoimmune encephalomyelitis (EAE; a model of multiple sclerosis), colitis, ulcerative colitis, Crohn's disease, inflammatory bowel disease, skin disease, asthma and arthritis.

Accordingly, compounds that inhibit TL1A activity are desirable, e.g., for their therapeutic, prophylactic, diagnostic and prognostic uses.

### SUMMARY OF THE INVENTION

Provided herein is a recombinant antibody comprising a heavy chain variable region CDR1 comprising the amino acid sequence of SEQ ID NO:15, a heavy chain variable region CDR2 comprising the amino acid sequence of SEQ ID NO:28, a heavy chain variable region CDR3 comprising the amino acid sequence of SEQ ID NO:17, a light chain variable region CDR1 comprising the amino acid sequence of SEQ ID NO:29, a light chain variable region CDR2 comprising the amino acid sequence of SEQ ID NO:19, and a light chain variable region CDR3 comprising the amino acid sequence of SEQ ID NO:30, provided that when the heavy chain variable region comprises the amino acid sequence of SEQ ID NO:1, the light chain variable region does not comprise the amino acid sequence of SEQ ID NO:2.

In some aspects, the antibody comprises a heavy chain variable region CDR2 comprising the amino acid sequence of SEQ ID NO:16, and a light chain variable region CDR3 comprising the amino acid sequence of SEQ ID NO:22. In some aspects, the antibody comprises a heavy chain variable region CDR2 comprising the amino acid sequence of SEQ ID NO:21, and a light chain variable region CDR3 comprising the amino acid sequence of SEQ ID NO:20. In some aspects, the antibody comprises a heavy chain variable region CDR2 comprising the amino acid sequence of SEQ ID NO:16, a light chain variable region CDR1 comprising the amino acid sequence of SEQ ID NO:23, and a light chain variable region CDR3 comprising the amino acid sequence of SEQ ID NO:22. In some aspects, the antibody comprises a heavy chain variable region CDR2 comprising the amino acid sequence of SEQ ID NO:16, a light chain variable region CDR1 comprising the amino acid sequence of SEQ ID NO:24, and a light chain variable region CDR3 comprising the amino acid sequence of SEQ ID NO:22. In some aspects, the antibody comprises a heavy chain variable region CDR2 comprising the amino acid sequence of SEQ ID NO:16, a light chain variable region CDR1 comprising the amino acid sequence of SEQ ID NO:25, and a light chain variable region CDR3 comprising the amino acid sequence of SEQ ID NO:22. In some aspects, the antibody comprises a heavy chain variable region CDR2 comprising the amino acid sequence of SEQ ID NO:16, a light chain variable region CDR1 comprising the amino acid sequence of SEQ ID NO:26, and a light chain variable region CDR3 comprising the amino acid sequence of SEQ ID NO:22. In some aspects, the antibody comprises a heavy chain variable region CDR2 comprising the amino acid sequence of SEQ ID NO:21 and a light chain variable region CDR3 comprising the amino acid sequence of SEQ ID NO:22. In some aspects, the antibody comprises a heavy chain variable region CDR2 comprising the amino acid sequence of SEQ ID NO:21, a light chain variable region CDR1 comprising the amino acid sequence of SEQ ID NO:24, and a light chain variable region CDR3 comprising the amino acid sequence of SEQ ID NO:22. In some aspects, the antibody comprises a heavy chain variable region CDR2 comprising the amino acid sequence of SEQ ID NO:21, a light chain variable region CDR1 comprising the amino acid sequence of SEQ ID NO:25, and a light chain variable region CDR3 comprising the amino acid sequence of SEQ ID NO:22. In some aspects, the antibody comprises a heavy chain variable region CDR2 comprising the amino acid sequence of SEQ ID NO:21, a light chain variable region CDR1 comprising the amino acid sequence of SEQ ID NO:26, and a light chain variable region CDR3 comprising the amino acid sequence of SEQ ID NO:22. In some aspects, the antibody comprises a heavy chain variable region CDR2 comprising the amino acid sequence of SEQ ID NO:21, and a light chain variable region CDR3 comprising the amino acid sequence of SEQ ID NO:27.

Also provided herein is a recombinant antibody comprising a heavy chain variable region comprising the amino acid sequence of SEQ ID NO:1 or SEQ ID NO:3 and a light chain variable region comprising the amino acid sequence of SEQ ID NO:14, provided that when the heavy chain variable region comprises the amino acid sequence of SEQ ID NO:1, the light chain variable region does not comprise the amino acid sequence of SEQ ID NO:2. In some aspects, the antibody comprises a heavy chain variable region comprising the amino acid sequence of SEQ ID NO:1, and a light chain variable region comprising the amino acid sequence of SEQ ID NO:4. In some aspects, the antibody comprises a heavy chain variable region comprising the amino acid sequence of SEQ ID NO:3, and a light chain variable region comprising the amino acid sequence of SEQ ID NO:2. In some aspects, the antibody comprises a heavy chain variable region comprising the amino acid sequence of SEQ ID NO:1, and a light chain variable region comprising the amino acid sequence of SEQ ID NO:5. In some aspects, the antibody comprises a heavy chain variable region comprising the amino acid sequence of SEQ ID NO:1, and a light chain variable region comprising the amino acid sequence of SEQ ID NO:6. In some aspects, the antibody comprises a heavy chain variable region comprising the amino acid sequence of SEQ ID NO:1, and a light chain variable region comprising the amino acid sequence of SEQ ID NO:7. In some aspects, the antibody comprises a heavy chain variable region comprising the amino acid sequence of SEQ ID NO:1, and a light chain variable region comprising the amino acid sequence of SEQ ID NO:8. In some aspects, the antibody comprises a heavy chain variable region comprising the amino acid sequence of SEQ ID NO:3, and a light chain variable region comprising the amino acid sequence of SEQ ID NO:4. In some aspects, the antibody comprises a heavy chain variable region comprising the amino acid sequence of SEQ ID NO:3, and a light chain variable region comprising the amino acid sequence of SEQ ID NO:6. In some aspects, the antibody comprises a heavy chain variable region comprising the amino acid sequence of SEQ ID NO:3, and a light chain variable region comprising the amino acid sequence of SEQ ID NO:7. In some aspects, the antibody comprises a heavy chain variable region comprising the amino acid sequence of SEQ ID NO:3, and a light chain variable region comprising the amino acid sequence of SEQ ID NO:8. In some aspects, the antibody comprises a heavy chain variable region comprising the amino acid sequence of SEQ ID NO:3, and a light chain variable region comprising the amino acid sequence of SEQ ID NO:10.

In some aspects, the antibody comprises a heavy chain comprising the amino acid sequence of SEQ ID NO:60. In some aspects, the antibody comprises a light chain comprising the amino acid sequence of SEQ ID NO:61. In some aspects, the antibody comprises a heavy chain comprising the amino acid sequence of SEQ ID NO:60 and a light chain comprising the amino acid sequence of SEQ ID NO:61.

Such recombinant antibodies preferably are full length, and preferably are monoclonal. Such recombinant antibodies bind to TL1A with enhanced affinity relative to an antibody having a heavy chain variable region comprising the amino acid sequence of SEQ ID NO:1 and a light chain variable region comprising the amino acid sequence of SEQ ID NO:2. Such recombinant antibodies have enhanced potency relative to an antibody having a heavy chain variable region comprising the amino acid sequence of SEQ ID NO:1 and a light chain variable region comprising the amino acid sequence of SEQ ID NO:2. The enhanced potency may be at least about 10-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 12-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 13-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 15-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 20-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 25-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 27-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 40-fold greater potency relative to an antibody having a heavy chain variable region comprising the amino acid sequence of SEQ ID NO:1 and a light chain variable region comprising the amino acid sequence of SEQ ID NO:2. Fold-enhancement of potency may be determined according to a TL1A-induced caspase potency assay in TF-1 cells.

Such recombinant antibodies may comprise a human IgG1 heavy chain constant region, a human IgG2 heavy chain constant region, or a human IgG4 heavy chain constant region, or any allotypes thereof. The human IgG1 heavy chain constant region may comprise SEQ ID NO:42, or SEQ ID NO:43 (human IgG1 ΔK), or SEQ ID NO:44 (human IgG1 with YTE), or SEQ ID NO:64 (human IgG1 with YTE and ΔK), or SEQ ID NO:63 (human IgG1 with L234A, L235A, G237A) or SEQ ID NO:62 (human IgG1 with L234A, L235A, G237A and ΔK), or SEQ ID NO:65 (human IgG1 with L235A and G237A) or SEQ ID NO:66 (human IgG1 with L235A, G237A and ΔK). The human IgG2 heavy chain constant region may comprise SEQ ID NO:67, or SEQ ID NO:70 (human IgG2 ΔK), or SEQ ID NO:71 (human IgG2 with A330S, P331S), or SEQ ID NO:68 (human IgG2 with A330S, P331S and ΔK). The human IgG4 heavy chain IgG4 constant region may comprise SEQ ID NO:45, or SEQ ID NO:46 (human IgG4 with S228P and ΔK), or SEQ ID NO:47 (human IgG4 with S228P and YTE), or SEQ ID NO:69 (human IgG4 with S228P, YTE and ΔK). It will be understood that an IgG4 heavy chain could be used without the stabilizing substitution S228P (e.g., IgG4 with YTE alone, or IgG4 with YTE and ΔK, or IgG4 with ΔK alone).

The recombinant antibodies may comprise a human lambda light chain constant region or an allotype thereof. The human light chain lambda constant region may comprise SEQ ID NO:48.

Such recombinant antibodies bind to human TL1A, and may bind to the TL1A of a non-human primate, or the TL1A of a non-human mammal such as a mouse, rat, guinea pig, cat, dog, rabbit, or pig.

Such recombinant antibodies may be used in a method for treating a respiratory tract disease, a method for treating a gastrointestinal disease, a method of treating a skin disease, or a method of treating arthritis, or may be for use in the treatment of a respiratory tract disease, a gastrointestinal disease, a skin disease, or arthritis, or may be for use in the manufacture of a medicament for the treatment of a respiratory tract disease, a gastrointestinal disease, a skin disease, or arthritis. The respiratory tract disease may comprise one or more of asthma, chronic obstructive pulmonary disease (COPD), pulmonary fibrosis, pulmonary sarcoidosis, allergic rhinitis, or cystic fibrosis. The gastrointestinal disease may comprise one or more of inflammatory bowel disease, Crohn's disease, colitis, ulcerative colitis, eosinophilic esophagitis, or irritable bowel syndrome, or a gastrointestinal disease or condition associated with cystic fibrosis. The arthritis may comprise rheumatoid arthritis. The skin disease may comprise one or more of atopic dermatitis, eczema, and scleroderma.

Human subjects, non-human primate subjects, or non-human mammalian subjects in need of such treatments may be treated with the antibodies or a composition comprising the antibodies, for example, by administering the antibodies or composition thereof to the subject. Administration may be parenteral, for example, subcutaneous and/or intravenous.

Such recombinant antibodies may be used in a method for detecting TL1A on the surface of peripheral blood mononuclear cells (PBMCs). The methods comprise contacting an antibody that binds to TL1A as described or exemplified herein with PBMCs obtained from a subject, and detecting the antibody bound to TL1A on the surface of the PBMCs. The methods may further comprise quantifying the level of TL1A on the PBMCs. The methods may further comprise obtaining the PBMCs from the subject.

Such recombinant antibodies may be used in a method for detecting TL1A in blood serum. The methods comprise contacting an antibody that binds to TL1A as described or exemplified herein with blood serum obtained from a subject, and detecting the antibody bound to TL1A in the serum. The methods may further comprise quantifying the level of TL1A in the blood serum. The methods may further comprise obtaining the serum from blood obtained from the subject. The methods may further comprise obtaining blood from the subject.

Polynucleotides encoding one or more of the heavy chain variable region and the light chain variable region of such antibodies are provided. The polynucleotides may further encode a heavy chain constant region and/or a light chain constant region.

In some aspects, a polynucleotide comprises a nucleic acid sequence encoding an antibody heavy chain variable region comprising the amino acid sequence of SEQ ID NO:3, for example, a nucleic acid sequence comprising SEQ ID NO:51 or SEQ ID NO:58. In some aspects, the polynucleotide comprises a nucleic acid sequence encoding an antibody light chain variable region comprising the amino acid sequence of SEQ ID NO:2, for example, a nucleic acid sequence comprising SEQ ID NO:50. In some aspects, the polynucleotide comprises a nucleic acid sequence encoding an antibody light chain variable region comprising the amino acid sequence of SEQ ID NO:4, for example, a nucleic acid sequence comprising SEQ ID NO:52 or SEQ ID NO:59. In some aspects, the polynucleotide comprises a nucleic acid sequence encoding an antibody light chain variable region comprising the amino acid sequence of SEQ ID NO:6, for example, a nucleic acid sequence comprising SEQ ID NO:54. In some aspects, the polynucleotide comprises a nucleic acid sequence encoding an antibody light chain variable region comprising the amino acid sequence of SEQ ID NO:7, for example, a nucleic acid sequence comprising SEQ ID NO:55. In some aspects, the polynucleotide comprises a nucleic acid sequence encoding an antibody light chain variable region comprising the amino acid sequence of SEQ ID NO:8, for example, a nucleic acid sequence comprising SEQ ID NO:56. In some aspects, the polynucleotide comprises a nucleic acid sequence encoding an antibody light chain variable region comprising the amino acid sequence of SEQ ID NO:10, for example, a nucleic acid sequence comprising SEQ ID NO:57.

In some aspects, the polynucleotide comprises a nucleic acid sequence encoding an antibody heavy chain variable region comprising the amino acid sequence of SEQ ID NO:1, for example, a nucleic acid sequence comprising SEQ ID NO:49. In some aspects, the polynucleotide comprises a nucleic acid sequence encoding an antibody light chain variable region comprising the amino acid sequence of SEQ ID NO:4, for example, a nucleic acid sequence comprising SEQ ID NO:52. In some aspects, the polynucleotide comprises a nucleic acid sequence encoding an antibody light chain variable region comprising the amino acid sequence of SEQ ID NO:5, for example, a nucleic acid sequence comprising SEQ ID NO:53. In some aspects, the polynucleotide comprises a nucleic acid sequence encoding an antibody light chain variable region comprising the amino acid sequence of SEQ ID NO:6, for example, a nucleic acid sequence comprising SEQ ID NO:54. In some aspects, the polynucleotide comprises a nucleic acid sequence encoding an antibody light chain variable region comprising the amino acid sequence of SEQ ID NO:7, for example, a nucleic acid sequence comprising SEQ ID NO:55. In some aspects, the polynucleotide comprises a nucleic acid sequence encoding an antibody light chain variable region comprising the amino acid sequence of SEQ ID NO:8, for example, a nucleic acid sequence comprising SEQ ID NO:56.

Vectors comprising one or more of such polynucleotides are provided. Cells transformed with one or more such polynucleotides or such vectors are provided. Transformed cells may be mammalian, and preferably are mammalian expression host cells such as CHO cells, NSO cells, or HEK293 cells.

### BRIEF DESCRIPTION OF THE DRAWINGS

FIG. 1 shows the positions and identities of each of the single amino acid substitutions made to the CDR1 or CDR2 regions or to selected amino acid residues adjacent to CDR2 of the parent antibody (320-179) variable heavy chain. Boxed regions represent the CDRs according to the AbM numbering system.

FIG. 2 shows the positions and the identities of each of the single amino acid substitutions made to the CDR1 or CDR3 regions of the parent antibody (320-179) variable light chain. Boxed regions represent the CDRs according to the AbM numbering system.

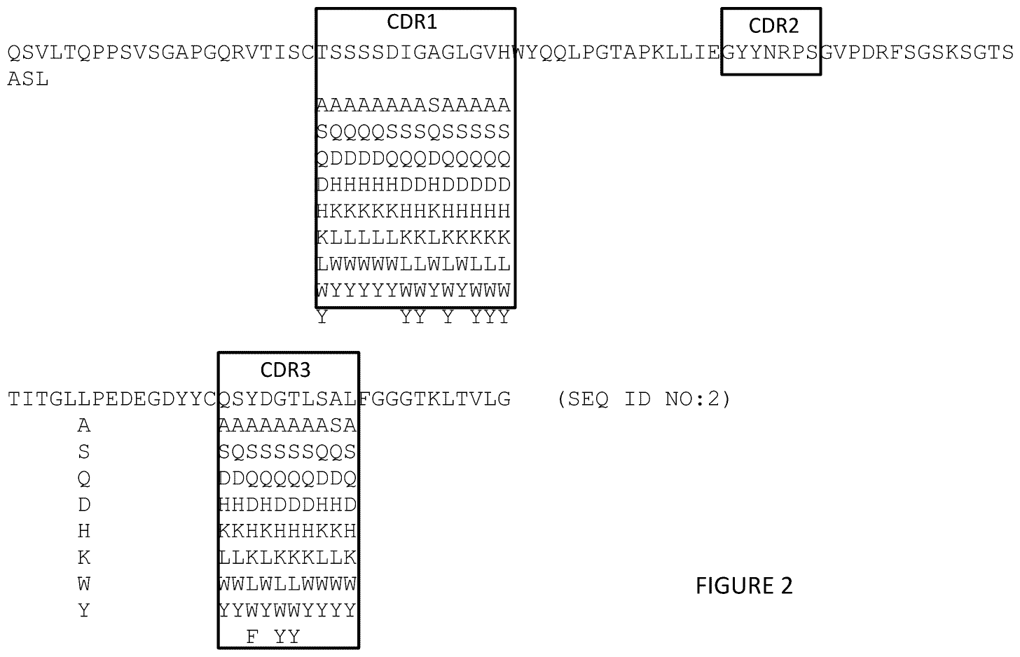

FIG. 3 shows an alignment of variant anti-TL1A-antibody heavy chains.

FIG. 4 shows an alignment of variant anti-TL1A-binding antibody light chains.

FIG. 5 shows a comparison of the TL1A dissociation phase of variant anti-TL1A antibodies, as measured by SPR.

FIG. 6 shows the results of TF-1 cell caspase potency assays with variant TL1A antibodies.

FIG. 7 shows the results of TF-1 cell caspase potency assays using various TL1A antibodies compared to the parent antibody, 320-179.

FIG. 8 shows that antibody 320-587 has superior TL1A potency in a TF-1 cell caspase potency assay, compared to other published anti-TL1A antibodies across several different experiments.

FIG. 9 shows various anti-TL1A antibodies that inhibit TL1A binding to DR3, as compared to an isotype control as measured in ELISA format.

FIG. 10 shows that the parent antibody, 320-179, does not inhibit TL1A binding to DcR3 while the variant anti-TL1A antibodies do inhibit the TL1A-DcR3 interaction.

FIG. 11 shows that antibody 320-587 cross reacts with and binds to TL1A of different species; the antibody bound to TL1A from all species tested.

FIG. 12 (A-D) shows the results of the administration of antibody 320-587 in a rat TNBS-induced colitis model, demonstrating that the antibody significantly ameliorates symptoms of colitis.

FIG. 13 shows that antibody 320-587 detects human TL1A secreted from human PBMCs stimulated with immune complexes, in an ELISA test.

FIG. 14 shows that antibody 320-587 detects a population of human PBMCs that express membrane TL1A on their surface, in a flow cytometry test.

FIG. 15 shows that rats having acute OVA-induced asthma treated with antibody 320-587 had significantly reduced eosinophils in the bronchoalveolar lavage fluid (BALF).

FIGS. 16A through 16D show treatment of guinea pigs having acute OVA-induced asthma treated with antibody 320-587 had improvements in (FIG. 16A) BALF eosinophils, (FIG. 16B) BALF macrophages, (FIG. 16C) Airways hyper responsiveness after early asthmatic reaction, and (FIG. 16D) Magnitude of early asthmatic reaction.

FIGS. 17A through 17E show treatment of rats having chronic OVA-induced asthma treated with antibody 320-587 had improvements in (FIG. 17A) BALF eosinophils, (FIG. 17B) BALF macrophages, (FIG. 17C) BALF IL-13, (FIG. 17D) goblet cell hyperplasia as assessed by PAS reactivity, and (FIG. 17E) mucosal thickening as assessed from H&E stained sections.

FIG. 18 shows the dose of ovalbumin required to double airways obstruction.

FIG. 19 shows treatment of rats having TNBS-induced colitis treated with antibody 320-587 had improvements in ulcer area fibrosis.

FIGS. 20A through 20E show a comparison of rats having TNBS-induced colitis with DNBS-induced colitis, treated with antibody 320-587 and the effects at 7 and 14 days, on (FIG. 20A) colon weight/length ratio, (FIG. 20B) colon fibrosis, (FIG. 20C) colon infiltrate, and (FIG. 20D) colon damage. FIG. 20E shows representative sections of ulcer area at 7 and 14 days.

FIGS. 21A through 21C show treatment of rats having DSS-induced colitis treatment with antibody 320-587 had improvements in (FIG. 21A) weight change during DSS administration, (FIG. 21B) Clinical scoring during DSS administration, and (FIG. 21C) colon weight/length ratio.

FIG. 22 shows changes in TL1A-induced intraperitoneal cytokines in response to 320-587 treatment.

### DETAILED DESCRIPTION OF THE INVENTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

The terms “subject” and “patient” are used interchangeably and include any animal. Mammals are preferred, including companion (e.g., cat, dog) and farm mammals (e.g., pig, horse, cow), as well as rodents, including mice, rabbits, and rats, guinea pigs, and other rodents. Non-human primates, such as cynomolgus monkeys, are more preferred, and human beings are highly preferred.

A molecule such as an antibody has been “isolated” if it has been altered and/or removed from its natural environment by the hand of a human being.

As used herein, the singular forms “a,” “an,” and “the” include plural referents unless expressly stated otherwise.

“Specificity” in the context of antibody-antigen interactions is not necessarily an absolute designation but may constitute a relative term signifying the degree of selectivity of an antibody for an antigen-positive cell compared to an antigen-negative cell. Specificity of an antibody for an antigen-positive cell is mediated by the variable regions of the antibody, and usually by the complementarity determining regions (CDRs) of the antibody. A construct may have from about 100 to about 1000-fold specificity for antigen-positive cells compared to antigen-negative cells.

As used herein, the term “recombinant” includes the expression from genes made by genetic engineering or otherwise by laboratory manipulation.

The disclosure provides variant anti-TL1A antibodies comprising a recombinantly altered heavy and/or light chain variable region of antibody 320-179, which variant antibodies specifically bind to TL1A. These 320-179 variant antibodies inhibit the capability of TL1A to interact with DR3 and, in some aspects, also with DcR3 and, further inhibit the signalling induced by the interaction of TL1A with DR3. These antibodies have enhanced potency relative to antibody 320-179. These antibodies have enhanced affinity for TL1A relative to antibody 320-179.

The enhanced potency may be at least about 10-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 12-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 13-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 15-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 20-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 25-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 27-fold greater potency relative to an antibody having a heavy chain variable region comprising the amino acid sequence of SEQ ID NO:1 and a light chain variable region comprising the amino acid sequence of SEQ ID NO:2. The enhanced potency may be at least about 40-fold greater potency relative to an antibody having a heavy chain variable region comprising the amino acid sequence of SEQ ID NO:1 and a light chain variable region comprising the amino acid sequence of SEQ ID NO:2. Fold-enhancement of potency may be determined, for example, by measuring caspase release in TL1A-induced apoptosis in a TF-1 cell assay.

The 320-179 variant antibodies are recombinantly expressed, and specifically bind to TL1A. The parent antibody, 320-179, comprises a heavy chain variable region comprising the amino acid sequence of SEQ ID NO:1 and a light chain variable region comprising the amino acid sequence of SEQ ID NO:2. In some aspects, a 320-179 variant antibody comprises a heavy chain variable region comprising the amino acid sequence of SEQ ID NO:1 or SEQ ID NO:3 and a light chain variable region comprising the amino acid sequence of SEQ ID NO:14, provided that when the heavy chain variable region comprises the amino acid sequence of SEQ ID NO:1, the light chain variable region does not comprise the amino acid sequence of SEQ ID NO:2. The 320-179 variant antibody is capable of inhibiting the interaction of TL1A with DR3. The 320-179 variant antibody has enhanced potency relative to antibody 320-179 and/or has enhanced affinity for TL1A relative to antibody 320-179.

In some aspects, the heavy chain variable region comprises the amino acid sequence of SEQ ID NO:1, and the light chain variable region comprises the amino acid sequence of SEQ ID NO:4. In some aspects, the heavy chain variable region comprises the amino acid sequence of SEQ ID NO:3, and the light chain variable region comprises the amino acid sequence of SEQ ID NO:2. In some aspects, the heavy chain variable region comprises the amino acid sequence of SEQ ID NO:1, and the light chain variable region comprises the amino acid sequence of SEQ ID NO:5. In some aspects, the heavy chain variable region comprises the amino acid sequence of SEQ ID NO:1, and the light chain variable region comprises the amino acid sequence of SEQ ID NO:6. In some aspects, the heavy chain variable region comprises the amino acid sequence of SEQ ID NO:1, and the light chain variable region comprises the amino acid sequence of SEQ ID NO:7. In some aspects, the heavy chain variable region comprises the amino acid sequence of SEQ ID NO:1, and the light chain variable region comprises the amino acid sequence of SEQ ID NO:8. In some highly preferred aspects, the heavy chain variable region comprises the amino acid sequence of SEQ ID NO:3, and the light chain variable region comprises the amino acid sequence of SEQ ID NO:4. In some aspects, the heavy chain variable region comprises the amino acid sequence of SEQ ID NO:3, and the light chain variable region comprises the amino acid sequence of SEQ ID NO:6. In some aspects, the heavy chain variable region comprises the amino acid sequence of SEQ ID NO:3, and the light chain variable region comprises the amino acid sequence of SEQ ID NO:7. In some aspects, the heavy chain variable region comprises the amino acid sequence of SEQ ID NO:3, and the light chain variable region comprises the amino acid sequence of SEQ ID NO:8. In some aspects, the heavy chain variable region comprises the amino acid sequence of SEQ ID NO:3, and the light chain variable region comprises the amino acid sequence of SEQ ID NO:10.

In highly preferred aspects, the 320-179 variant antibody comprises a heavy chain variable region comprising the amino acid sequence of SEQ ID NO:3, and a light chain variable region comprising the amino acid sequence of SEQ ID NO:4, and specifically binds to TL1A. In some aspects, the heavy chain variable region of SEQ ID NO:3 is joined to a human IgG1 (ΔK) heavy chain constant region (e.g., SEQ ID NO:43) such that the heavy chain comprises SEQ ID NO:60. In some aspects, the light chain variable region of SEQ ID NO:4 is joined to a lambda human light chain constant region (e.g., SEQ ID NO:48) such that the light chain comprises SEQ ID NO:61. The 320-179 variant antibody is capable of inhibiting the interaction of TL1A with DR3. The variant antibody has enhanced potency relative to antibody 320-179 and/or has enhanced affinity for TL1A relative to antibody 320-179.

In some aspects, the 320-179 variant antibodies are recombinantly expressed and specifically bind to TL1A, and comprise a heavy chain variable region CDR1 comprising the amino acid sequence of SEQ ID NO:15, a heavy chain variable region CDR2 comprising the amino acid sequence of SEQ ID NO:28, and a heavy chain variable region CDR3 comprising the amino acid sequence of SEQ ID NO:17. The antibodies may comprise a light chain variable region CDR1 comprising the amino acid sequence of SEQ ID NO:29, a light chain variable region CDR2 comprising the amino acid sequence of SEQ ID NO:19, and a light chain variable region CDR3 comprising the amino acid sequence of SEQ ID NO:30. In aspects where the heavy chain variable region comprises the amino acid sequence of SEQ ID NO:1, the light chain variable region preferably does not comprise the amino acid sequence of SEQ ID NO:2. These 320-179 variant antibodies are capable of inhibiting the interaction of TL1A with DR3. These 320-179 variant antibodies have enhanced potency relative to antibody 320-179 and/or have enhanced affinity for TL1A relative to antibody 320-179.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:16, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:16, a heavy chain variable region CDR3 comprising the amino acid sequence of SEQ ID NO:17, a light chain variable region CDR1 comprising the amino acid sequence of SEQ ID NO:23, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:16, a heavy chain variable region CDR3 comprising the amino acid sequence of SEQ ID NO:17, a light chain variable region CDR1 comprising the amino acid sequence of SEQ ID NO:24, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:16, a heavy chain variable region CDR3 comprising the amino acid sequence of SEQ ID NO:17, a light chain variable region CDR1 comprising the amino acid sequence of SEQ ID NO:25, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:20.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:16, a heavy chain variable region CDR3 comprising the amino acid sequence of SEQ ID NO:17, a light chain variable region CDR1 comprising the amino acid sequence of SEQ ID NO:26, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:24, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:25, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:26, a light chain variable region CDR2 comprising the amino acid sequence of SEQ ID NO:19, and a light chain variable region CDR3 comprising the amino acid sequence of SEQ ID NO:22.

In some aspects, the antibodies specifically bind to TL1A and comprise a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:27.

In some aspects, the antibodies specifically bind to TL1A, and comprise a heavy chain variable region comprising the amino acid sequence of SEQ ID NO:1 and a light chain variable region, provided that the light chain variable region does not comprise the amino acid sequence of SEQ ID NO:2. In some aspects, the antibodies specifically bind to TL1A, and comprise a heavy chain variable region comprising the amino acid sequence of SEQ ID NO:3 and a light chain variable region or a light chain. The light chain variable region may further comprise a lambda constant region.

In some aspects, the antibodies specifically bind to TL1A, and comprise a light chain variable region comprising the amino acid sequence of SEQ ID NO:2 and a heavy chain variable region, provided that the heavy chain variable region does not comprise the amino acid sequence of SEQ ID NO:1. In some aspects, the antibodies specifically bind to TL1A, and comprise a light chain variable region comprising the amino acid sequence of SEQ ID NO:4 and a heavy chain variable region. In some aspects, the antibodies specifically bind to TL1A, and comprise a light chain variable region comprising the amino acid sequence of SEQ ID NO:5 and a heavy chain variable region. In some aspects, the antibodies specifically bind to TL1A, and comprise a light chain variable region comprising the amino acid sequence of SEQ ID NO:6 and a heavy chain variable region. In some aspects, the antibodies specifically bind to TL1A, and comprise a light chain variable region comprising the amino acid sequence of SEQ ID NO:7 and a heavy chain variable region. In some aspects, the antibodies specifically bind to TL1A, and comprise a light chain variable region comprising the amino acid sequence of SEQ ID NO:8 and a heavy chain variable region. In some aspects, the antibodies specifically bind to TL1A, and comprise a light chain variable region comprising the amino acid sequence of SEQ ID NO:9 and a heavy chain variable region. In some aspects, the antibodies specifically bind to TL1A, and comprise a light chain variable region comprising the amino acid sequence of SEQ ID NO:10 and a heavy chain variable region. In some aspects, the antibodies specifically bind to TL1A, and comprise a light chain variable region comprising the amino acid sequence of SEQ ID NO:11 and a heavy chain variable region. In some aspects, the antibodies specifically bind to TL1A, and comprise a light chain variable region comprising the amino acid sequence of SEQ ID NO:12 and a heavy chain variable region. In some aspects, the antibodies specifically bind to TL1A, and comprise a light chain variable region comprising the amino acid sequence of SEQ ID NO:13 and a heavy chain variable region. In some aspects, the antibodies specifically bind to TL1A, and comprise a light chain variable region comprising the amino acid sequence of SEQ ID NO:14 and a heavy chain variable region, provided that if the light chain variable region comprises the amino acid sequence of SEQ ID NO:2, the heavy chain variable region does not comprise the amino acid sequence of SEQ ID NO:1. The heavy chain variable region may further comprise a heavy chain constant region, including any IgG1, IgG2, or IgG4 heavy chain constant region amino acid sequence described or exemplified herein.

The 320-179-variant antibodies specifically bind to TL1A. The antibodies bind to human TL1A, and may bind to one or more of cynomolgus monkey TL1A, mouse TL1A, rat TL1A, guinea pig TL1A, cat TL1A, dog TL1A, pig TL1A, or rabbit TL1A. In some aspects, the antibodies may bind to TL1A of multiple different species, for example, if the epitope is shared. In some aspects, human TL1A comprises the amino acid sequence of SEQ ID NO:31, SEQ ID NO:32, or SEQ ID NO:33. In some aspects, cynomolgus monkey TL1A comprises the amino acid sequence of SEQ ID NO:34. In some aspects, mouse TL1A comprises the amino acid sequence of SEQ ID NO:35. In some aspects, rat TL1A comprises the amino acid sequence of SEQ ID NO:36. In some aspects, guinea pig TL1A comprises the amino acid sequence of SEQ ID NO:37. In some aspects, cat TL1A comprises the amino acid sequence of SEQ ID NO:38. In some aspects, pig TL1A comprises the amino acid sequence of SEQ ID NO:39. In some aspects, rabbit TL1A comprises the amino acid sequence of SEQ ID NO:40. In some aspects, dog TL1A comprises the amino acid sequence of SEQ ID NO:41.

The 320-179-variant antibodies have a binding affinity for an epitope on TL1A that includes an equilibrium dissociation constant (K_{D}), which can be measured according to a kinetic exclusion assay, such as a KINEXA® assay (Sapidyne Instruments Inc., Boise, ID). The K_{D} for TL1A binding determined from a kinetic exclusion assay is preferably less than about 1000 pM. In some aspects, the K_{D} for TL1A binding determined from a kinetic exclusion assay is less than about 500 pM, or less than about 400 pM, or less than about 300pM, or less than about 200 pM. In some preferred aspects, the K_{D} for TL1A binding determined from a kinetic exclusion assay is less than about 100 pM.

The K_{D} for TL1A binding determined from a kinetic exclusion assay may be from about 10 pM to about 100 pM. The K_{D} for TL1A binding determined from a kinetic exclusion assay may be from about 25 pM to about 75 pM. The K_{D} for TL1A binding determined from a kinetic exclusion assay may be from about 30 pM to about 60 pM. The K_{D} for TL1A binding determined from a kinetic exclusion assay may be from about 30 pM to about 50 pM. The K_{D} for TL1A binding determined from a kinetic exclusion assay may be from about 35 pM to about 50 pM. The K_{D} for TL1A binding determined from a kinetic exclusion assay may be from about 36 pM to about 46 pM. The K_{D} for TL1A binding determined from a kinetic exclusion assay may be from about 38 PM to about 44 pM. The K_{D} for TL1A binding determined from a kinetic exclusion assay may be from about 39 PM to about 43 pM. The K_{D} for TL1A binding determined from a kinetic exclusion assay may be from about 40 pM to about 45 pM. The K_{D} for TL1A binding determined from a kinetic exclusion assay may be from about 35 pM to about 42 pM. The K_{D} for TL1A binding determined from a kinetic exclusion assay may about 40 pM. The K_{D} for TL1A binding determined from a kinetic exclusion assay may about 41 pM. The K_{D} for TL1A binding determined from a kinetic exclusion assay may about 42 pM. The kinetic exclusion assay may use the antibody molecule or TL1A molecule as the constant binding partner, and the other molecule as the titrant.

The 320-179-variant anti-TL1A antibodies are preferably capable of binding to TL1A-positive cells. The antibody may bind to a TL1A-positive cell with an EC50 value of less than about 100 nM. The antibody may bind to a TL1A-positive cell with an EC50 value of less than about 75 nM. The antibody may bind to a TL1A-positive cell with an EC50 value of less than about 50 nM. The antibody may bind to a TL1A-positive cell with an EC50 value of less than about 30 nM. The antibody may bind to a TL1A-positive cell with an EC50 value of less than about 25 nM. The antibody may bind to a TL1A-positive cell with an EC50 value of less than about 20 nM. The antibody may bind to a TL1A-positive cell with an EC50 value of less than about 18 nM. The antibody may bind to a TL1A-positive cell with an EC50 value of less than about 15 nM. The antibody may bind to a TL1A-positive cell with an EC50 value of less than about 13 nM. The antibody may bind to a TL1A-positive cell with an EC50 value of less than about 10 nM.

The 320-179-variant antibodies preferably are monoclonal, and more preferably are full length antibodies comprising two heavy chains and two light chains. In some aspects, the antibodies comprise derivatives or fragments or portions of antibodies that retain the antigen-binding specificity, and also preferably retain most or all of the affinity, of the 320-179 parent antibody molecule (e.g., for TL1A). For example, derivatives may comprise at least one variable region (either a heavy chain or light chain variable region). Other examples of suitable antibody derivatives and fragments include, without limitation, antibodies with polyepitopic specificity, bispecific antibodies, multi-specific antibodies, diabodies, single-chain molecules, as well as FAb, F(Ab′)2, Fd, Fabc, and Fv molecules, single chain (Sc) antibodies, single chain Fv antibodies (scFv), individual antibody light chains, individual antibody heavy chains, fusions between antibody chains and other molecules, heavy chain monomers or dimers, light chain monomers or dimers, dimers consisting of one heavy and one light chain, and other multimers. Single chain Fv antibodies may be multi-valent. All antibody isotypes may be used to produce antibody derivatives, fragments, and portions. Antibody derivatives, fragments, and/or portions may be recombinantly produced and expressed by any cell type, prokaryotic or eukaryotic.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Typically, the antigen binding properties of an antibody are less likely to be disturbed by changes to FR sequences than by changes to the CDR sequences. Immunoglobulin molecules can be of any type (e.g., IgG, IgE, IgM, IgD, IgA and lgY), class (e.g., IgG1, IgG2, IgG3, IgG4, IgA1 and IgA2) or subclass.

The 320-179-variant antibodies are fully human. Fully human antibodies are those where the whole molecule is human or otherwise of human origin, or includes an amino acid sequence identical to a human form of the antibody. Fully human antibodies include those obtained from a human V gene library, for example, where human genes encoding variable regions of antibodies are recombinantly expressed. Fully human antibodies may be expressed in other organisms (e.g., mice and xenomouse technology) or cells from other organisms transformed with genes encoding human antibodies. Fully human antibodies may nevertheless include amino acid residues not encoded by human sequences, e.g., mutations introduced by random or site directed mutations.

In some aspects, the 320-179-variant antibodies may comprise non-immunoglobulin derived protein frameworks. For example, reference may be made to (Ku & Schutz, 1995, Proc. Natl. Acad. Sci. USA 92:6552-6556) which describes a four-helix bundle protein cytochrome b562 having two loops randomized to create CDRs, which have been selected for antigen binding.

The 320-179-variant antibodies may comprise post-translational modifications or moieties, which may impact antibody activity or stability. These modifications or moieties include, but are not limited to, methylated, acetylated, glycosylated, sulfated, phosphorylated, carboxylated, and amidated moieties and other moieties that are well known in the art. Moieties include any chemical group or combinations of groups commonly found on immunoglobulin molecules in nature or otherwise added to antibodies by recombinant expression systems, including prokaryotic and eukaryotic expression systems.

Examples of side chain modifications contemplated by the disclosure include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal. The carboxyl group may be modified by carbodiimide activation via O-acylisourea formation followed by subsequent derivation, for example, to a corresponding amide. Sulfydryl groups may be modified by methods such as carboxymethylation with iodoacetic acid or iodoacetamide; performic acid oxidation to cysteic acid; formation of mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH. Tryptophan residues may be modified by, for example, oxidation with N-bromosuccinimide or alkylation of the indole ring with 2-hydroxy-5-nitrobenzyl bromide or sulfenyl halides. Tyrosine residues on the other hand, may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate.

The 320-179-variant antibodies may include modifications that modulate serum half-life and biodistribution, including without limitation, modifications that modulate the antibody's interaction with the neonatal Fc receptor (FcRn), a receptor with a key role in protecting IgG from catabolismand maintaining high serum antibody concentration. Serum half-life modulating modifications may occur in the Fc region of IgG1, IgG2, or IgG4, including the triple substitution of M252Y/S254T/T256E (the “YTE” substitutions, with numbering according to the EU numbering system (Edelman, G.M. et al. (1969) Proc. Natl. Acad. USA 63, 78-85)), as described in U.S. Pat. No. 7,083,784. Other substitutions may occur at positions 250 and 428, see e.g., U.S. Pat. No 7,217,797, as well as at positions 307, 380 and 434, see, e.g., PCT Publ. No. WO 00/042072. Examples of constant domain amino acid substitutions which modulate binding to Fc receptors and subsequent function mediated by these receptors, including FcRn binding and serum half-life, are described in U.S. Publ. Nos. 2009/0142340, 2009/0068175, and 2009/0092599. Antibodies of any class may have the heavy chain C-terminal lysine omitted or removed to reduce heterogeneity (ΔK). The substitution of S228P (EU numbering) in the human IgG4 can stabilize antibody Fab-arm exchange in vivo (Labrin et al. (2009) Nature Biotechnology 27:8; 767-773), and this substitution may be present at the same time as the YTE and/or ΔK modifications.

The 320-179-variant antibodies comprise human constant domains. The heavy chain constant domains preferably are human IgG1, IgG2, or IgG4 constant domains. The light chain constant domains preferably are human lambda constant domains. A suitable human lambda domain comprises SEQ ID NO:48.

Human heavy chain IgG1 constant regions that may be used with the 320-179 variant antibodies may be selected from among human IgG1 (SEQ ID NO:42), human IgG1 (ΔK) (SEQ ID NO:43), human IgG1 252Y/254T/256E (SEQ ID NO:44), human IgG1 252Y/254T/256E (ΔK) (SEQ ID NO:64), human IgG1 L234A/L235A/G237A (SEQ ID NO:63), human IgG1L234A/L235A/G237A (ΔK) (SEQ ID NO:62), human IgG1 L235A/G237A (SEQ ID NO:65), and human IgG1 L235A/G237A (ΔK) (SEQ ID NO:66). Human heavy chain IgG2 constant regions that may be used with the 320-179 variant antibodies may be selected from among human IgG2 with or without ΔK (SEQ ID NO:67 and SEQ ID NO:70) and human IgG2 A330S/P331S with or without (ΔK) (SEQ ID NO:71 and SEQ ID NO:68). Human heavy chain IgG4 constant regions that may be used with the 320-179 variant antibodies may be selected from among human IgG4 S228P (SEQ ID NO:45), human IgG4 S228P (ΔK) (SEQ ID NO:46), human IgG4 228P/252Y/254T/256E (SEQ ID NO:47), and human IgG4 228P/252Y/254T/256E (ΔK) (SEQ ID NO:69).

The 320-179-variant antibodies may be labelled, bound, or conjugated to any chemical or biomolecule moieties. Labelled antibodies may find use in therapeutic, diagnostic, or basic research applications. Such labels/conjugates can be detectable, such as fluorochromes, electrochemiluminescent probes, quantum dots, radiolabels, enzymes, fluorescent proteins, luminescent proteins, and biotin. The labels/conjugates may be chemotherapeutic agents, toxins, isotopes, and other agents used for treating conditions such as the killing of cancer cells. Chemotherapeutic agents may be any which are suitable for the purpose for which the antibody is being used.

The antibodies may be derivatized by known protecting/blocking groups to prevent proteolytic cleavage or enhance activity or stability.

Polynucleotide sequences that encode antibodies and their subdomains (e.g., FRs and CDRs) are featured in the disclosure. Polynucleotides include, but are not limited to, RNA, DNA, cDNA, hybrids of RNA and DNA, and single, double, or triple stranded strands of RNA, DNA, or hybrids thereof. Polynucleotides may comprise a nucleic acid sequence encoding the heavy chain variable region and/or the light chain variable region of a 320-179 variant antibody as described or exemplified herein. Complements of the polynucleotide sequences are also within the scope of the disclosure.

A polynucleotide may comprise a nucleic acid sequence encoding an antibody heavy chain variable region comprising the amino acid sequence of SEQ ID NO:3. A polynucleotide encoding the amino acid sequence of SEQ ID NO:3 may comprise the nucleic acid sequence of SEQ ID NO:51.

A polynucleotide may comprise a nucleic acid sequence encoding an antibody heavy chain variable region comprising the amino acid sequence of SEQ ID NO:1. A polynucleotide encoding the amino acid sequence of SEQ ID NO:1 may comprise the nucleic acid sequence of SEQ ID NO:49.

A polynucleotide may comprise a nucleic acid sequence encoding an antibody light chain variable region comprising the amino acid sequence of SEQ ID NO:2. A polynucleotide encoding the amino acid sequence of SEQ ID NO:2 may comprise the nucleic acid sequence of SEQ ID NO:50.

A polynucleotide may comprise a nucleic acid sequence encoding an antibody light chain variable region comprising the amino acid sequence of SEQ ID NO:4. A polynucleotide encoding the amino acid sequence of SEQ ID NO:4 may comprise the nucleic acid sequence of SEQ ID NO:52.

A polynucleotide may comprise a nucleic acid sequence encoding an antibody light chain variable region comprising the amino acid sequence of SEQ ID NO:5. A polynucleotide encoding the amino acid sequence of SEQ ID NO:5 may comprise the nucleic acid sequence of SEQ ID NO:53.

A polynucleotide may comprise a nucleic acid sequence encoding an antibody light chain variable region comprising the amino acid sequence of SEQ ID NO:6. A polynucleotide encoding the amino acid sequence of SEQ ID NO:6 may comprise the nucleic acid sequence of SEQ ID NO:54.

A polynucleotide may comprise a nucleic acid sequence encoding an antibody light chain variable region comprising the amino acid sequence of SEQ ID NO:7. A polynucleotide encoding the amino acid sequence of SEQ ID NO:7 may comprise the nucleic acid sequence of SEQ ID NO:55.

A polynucleotide may comprise a nucleic acid sequence encoding an antibody light chain variable region comprising the amino acid sequence of SEQ ID NO:8. A polynucleotide encoding the amino acid sequence of SEQ ID NO:8 may comprise the nucleic acid sequence of SEQ ID NO:56.

A polynucleotide may comprise a nucleic acid sequence encoding an antibody light chain variable region comprising the amino acid sequence of SEQ ID NO:10. A polynucleotide encoding the amino acid sequence of SEQ ID NO:10 may comprise the nucleic acid sequence of SEQ ID NO:57.

In some aspects, a polynucleotide comprises a first nucleic acid sequence encoding an antibody heavy chain variable region and a second nucleic acid sequence encoding an antibody light chain variable region. A first nucleic acid sequence may encode an antibody heavy chain variable region comprising the amino acid sequence of SEQ ID NO:3 and a light chain variable region comprising the amino acid sequence of SEQ ID NO:2. A polynucleotide encoding the amino acid sequence of SEQ ID NO:3 may comprise the nucleic acid sequence of SEQ ID NO:51 and a polynucleotide encoding the amino acid sequence of SEQ ID NO:2 may comprise the nucleic acid sequence of SEQ ID NO:50.

A first nucleic acid sequence may encode an antibody heavy chain variable region comprising the amino acid sequence of SEQ ID NO:3 and a light chain variable region comprising the amino acid sequence of SEQ ID NO:4. A polynucleotide encoding the amino acid sequence of SEQ ID NO:3 may comprise the nucleic acid sequence of SEQ ID NO:51 and a polynucleotide encoding the amino acid sequence of SEQ ID NO:4 may comprise the nucleic acid sequence of SEQ ID NO:52.

A first nucleic acid sequence may encode an antibody heavy chain variable region comprising the amino acid sequence of SEQ ID NO:3 and a light chain variable region comprising the amino acid sequence of SEQ ID NO:6. A polynucleotide encoding the amino acid sequence of SEQ ID NO:3 may comprise the nucleic acid sequence of SEQ ID NO:51 and a polynucleotide encoding the amino acid sequence of SEQ ID NO:6 may comprise the nucleic acid sequence of SEQ ID NO:54.

A first nucleic acid sequence may encode an antibody heavy chain variable region comprising the amino acid sequence of SEQ ID NO:3 and a light chain variable region comprising the amino acid sequence of SEQ ID NO:7. A polynucleotide encoding the amino acid sequence of SEQ ID NO:3 may comprise the nucleic acid sequence of SEQ ID NO:51 and a polynucleotide encoding the amino acid sequence of SEQ ID NO:7 may comprise the nucleic acid sequence of SEQ ID NO:55.

A first nucleic acid sequence may encode an antibody heavy chain variable region comprising the amino acid sequence of SEQ ID NO:3 and a light chain variable region comprising the amino acid sequence of SEQ ID NO:8. A polynucleotide encoding the amino acid sequence of SEQ ID NO:3 may comprise the nucleic acid sequence of SEQ ID NO:51 and a polynucleotide encoding the amino acid sequence of SEQ ID NO:8 may comprise the nucleic acid sequence of SEQ ID NO:56.

A first nucleic acid sequence may encode an antibody heavy chain variable region comprising the amino acid sequence of SEQ ID NO:3 and a light chain variable region comprising the amino acid sequence of SEQ ID NO:10. A polynucleotide encoding the amino acid sequence of SEQ ID NO:3 may comprise the nucleic acid sequence of SEQ ID NO:51 and a polynucleotide encoding the amino acid sequence of SEQ ID NO:10 may comprise the nucleic acid sequence of SEQ ID NO:57.

A first nucleic acid sequence may encode an antibody heavy chain variable region comprising the amino acid sequence of SEQ ID NO:1 and a light chain variable region comprising the amino acid sequence of SEQ ID NO:4. A polynucleotide encoding the amino acid sequence of SEQ ID NO:1 may comprise the nucleic acid sequence of SEQ ID NO:49 and a polynucleotide encoding the amino acid sequence of SEQ ID NO:4 may comprise the nucleic acid sequence of SEQ ID NO:52.

A first nucleic acid sequence may encode an antibody heavy chain variable region comprising the amino acid sequence of SEQ ID NO:1 and a light chain variable region comprising the amino acid sequence of SEQ ID NO:5. A polynucleotide encoding the amino acid sequence of SEQ ID NO:1 may comprise the nucleic acid sequence of SEQ ID NO:49 and a polynucleotide encoding the amino acid sequence of SEQ ID NO:5 may comprise the nucleic acid sequence of SEQ ID NO:53.

A first nucleic acid sequence may encode an antibody heavy chain variable region comprising the amino acid sequence of SEQ ID NO:1 and a light chain variable region comprising the amino acid sequence of SEQ ID NO:6. A polynucleotide encoding the amino acid sequence of SEQ ID NO:1 may comprise the nucleic acid sequence of SEQ ID NO:49 and a polynucleotide encoding the amino acid sequence of SEQ ID NO:6 may comprise the nucleic acid sequence of SEQ ID NO:54.

A first nucleic acid sequence may encode an antibody heavy chain variable region comprising the amino acid sequence of SEQ ID NO:1 and a light chain variable region comprising the amino acid sequence of SEQ ID NO:7. A polynucleotide encoding the amino acid sequence of SEQ ID NO:1 may comprise the nucleic acid sequence of SEQ ID NO:49 and a polynucleotide encoding the amino acid sequence of SEQ ID NO:7 may comprise the nucleic acid sequence of SEQ ID NO:55.

A first nucleic acid sequence may encode an antibody heavy chain variable region comprising the amino acid sequence of SEQ ID NO:1 and a light chain variable region comprising the amino acid sequence of SEQ ID NO:8. A polynucleotide encoding the amino acid sequence of SEQ ID NO:1 may comprise the nucleic acid sequence of SEQ ID NO:49 and a polynucleotide encoding the amino acid sequence of SEQ ID NO:8 may comprise the nucleic acid sequence of SEQ ID NO:56.

In some aspects, a polynucleotide comprises a first nucleic acid sequence encoding an antibody heavy chain variable region and a second nucleic acid sequence encoding a heavy chain constant region. In preferred aspects, a polynucleotide comprises a first nucleic acid sequence encoding an antibody heavy chain variable region comprising the amino acid sequence of SEQ ID NO:3 and a second nucleic acid sequence encoding an IgG1 (ΔK) heavy chain constant region of SEQ ID NO:43, for example, a polynucleotide comprising the nucleic acid sequence of SEQ ID NO:58.

In some aspects, a polynucleotide comprises a first nucleic acid sequence encoding an antibody light chain variable region and a second nucleic acid sequence encoding a light chain constant region. In preferred aspects, a polynucleotide comprises a first nucleic acid sequence encoding an antibody light chain variable region comprising the amino acid sequence of SEQ ID NO:4 and a second nucleic acid sequence encoding a lambda light chain constant region of SEQ ID NO:48, for example, a polynucleotide comprising the nucleic acid sequence of SEQ ID NO:59.

Any of the polynucleotides described or exemplified herein may be comprised within a vector. Thus, vectors comprising polynucleotides are provided as part of the disclosure. The vectors may be expression vectors. Recombinant expression vectors containing a sequence encoding a polypeptide of interest are thus provided. The expression vector may contain one or more additional sequences, such as but not limited to regulatory sequences, a selection marker, a purification tag, or a polyadenylation signal. Such regulatory elements may include a transcriptional promoter, enhancers, mRNA ribosomal binding sites, or sequences that control the termination of transcription and translation.

Expression vectors, especially mammalian expression vectors, may include one or more nontranscribed elements, such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a specific host may also be incorporated.

The vectors may be used to transform any of a wide array of host cells well known to those of skill in the art, and preferably host cells capable of expressing antibodies. Vectors include without limitation, plasmids, phagemids, cosmids, bacmids, bacterial artificial chromosomes (BACs), yeast artificial chromosomes (YACs), and baculovirus, as well as other bacterial, eukaryotic, yeast, and viral vectors. Suitable host cells include without limitation CHO cells, NSO cells, HEK293 cells, or any eukaryotic stable cell line known or produced, and also include bacteria, yeast, and insect cells.

The antibodies may also be produced by hybridoma cells; methods to produce hybridomas being well known and established in the art.

The disclosure also provides compositions comprising the 320-179 variant antibodies. The compositions may comprise any of the antibodies described and/or exemplified herein and an acceptable carrier such as a pharmaceutically acceptable carrier. Suitable carriers include any media that does not interfere with the biological activity of the antibody and preferably is not toxic to a host to which it is administered. The compositions may be formulated for administration to a subject in any suitable dosage form.

The 320-179 variant antibodies may be used to treat a respiratory tract disease, a gastrointestinal disease, arthritis, or a skin disease in a subject. Thus, the disclosure features treatment methods. In general, the methods comprise administering a 320-179 variant antibody, or composition thereof, to a subject in need of treatment for a respiratory tract disease, gastrointestinal disease, arthritis, or a skin disease, such that the respiratory tract disease, gastrointestinal disease, arthritis, or skin disease is treated. The 320-179 variant antibody may comprise any antibody described or exemplified herein. Administering may comprise subcutaneously administering the antibody. Administering may comprise intravenously administering the antibody. The subject is preferably a human being. The subject may be a non-human primate such as a cynomolgus monkey, or may be a mammal such as a mouse, rat, guinea pig, cat, pig, rabbit, or dog.

In aspects where a respiratory tract disease is to be treated, the methods comprise administering a 320-179 variant antibody, or composition thereof, to a subject in need of treatment for a respiratory tract disease. The respiratory tract disease may comprise one or more of asthma, chronic obstructive pulmonary disease (COPD), pulmonary fibrosis, pulmonary sarcoidosis, allergic rhinitis, or cystic fibrosis. Thus, for example, in some aspects, the methods comprise administering a 320-179 variant antibody, or composition thereof, to a subject in need of treatment for asthma, such that the asthma is treated in the subject. In some aspects, the methods comprise administering a 320-179 variant antibody, or composition thereof, to a subject in need of treatment for COPD, such that the COPD is treated in the subject. In some aspects, the methods comprise administering a 320-179 variant antibody, or composition thereof, to a subject in need of treatment for pulmonary fibrosis, such that the pulmonary fibrosis is treated in the subject. In some aspects, the methods comprise administering a 320-179 variant antibody, or composition thereof, to a subject in need of treatment for pulmonary sarcoidosis, such that the pulmonary sarcoidosis is treated in the subject. In some aspects, the methods comprise administering a 320-179 variant antibody, or composition thereof, to a subject in need of treatment for allergic rhinitis, such that the allergic rhinitis is treated in the subject. In some aspects, the methods comprise administering a 320-179 variant antibody, or composition thereof, to a subject in need of treatment for cystic fibrosis, such that the cystic fibrosis is treated in the subject.

In aspects where a gastrointestinal disease is to be treated, the methods comprise administering a 320-179 variant antibody, or composition thereof, to a subject in need of treatment for a gastrointestinal disease. The gastrointestinal disease may comprise one or more of inflammatory bowel disease (IBD), Crohn's disease, colitis, ulcerative colitis, irritable bowel syndrome (IBS), eosinophilic esophagitis, or a gastrointestinal disease or condition associated with cystic fibrosis. Thus, for example, in some aspects, the methods comprise administering a 320-179 variant antibody, or composition thereof, to a subject in need of treatment for IBD, such that the IBD is treated in the subject. In some aspects, the methods comprise administering a 320-179 variant antibody, or composition thereof, to a subject in need of treatment for Crohn's disease, such that the Crohn's disease is treated in the subject. In some aspects, the methods comprise administering a 320-179 variant antibody, or composition thereof, to a subject in need of treatment for colitis, such that the colitis is treated in the subject. In some aspects, the methods comprise administering a 320-179 variant antibody, or composition thereof, to a subject in need of treatment for ulcerative colitis, such that the ulcerative colitis is treated in the subject. In some aspects, the methods comprise administering a 320-179 variant antibody, or composition thereof, to a subject in need of treatment for IBS, such that the IBS is treated in the subject. In some aspects, the methods comprise administering a 320-179 variant antibody, or composition thereof, to a subject in need of treatment for eosinophilic esophagitis, such that the eosinophilic esophagitis is treated in the subject. In some aspects, the methods comprise administering a 320-179 variant antibody, or composition thereof, to a subject in need of treatment for a gastrointestinal disease or condition associated with cystic fibrosis, such that the gastrointestinal disease or condition associated with cystic fibrosis is treated in the subject.

In aspects where arthritis is to be treated, the methods comprise administering a 320-179 variant antibody, or composition thereof, to a subject in need of treatment for arthritis. The arthritis may comprise rheumatoid arthritis. Thus, for example, in some aspects, the methods comprise administering a 320-179 variant antibody, or composition thereof, to a subject in need of treatment for rheumatoid arthritis, such that the rheumatoid arthritis is treated in the subject.

In aspects where a skin disease is to be treated, the methods comprise administering a 320-179 variant antibody, or composition thereof, to a subject in need of treatment for a skin disease. The skin disease may comprise one or more of atopic dermatitis, eczema, or scleroderma. Thus, for example, in some aspects, the methods comprise administering a 320-179 variant antibody, or composition thereof, to a subject in need of treatment for atopic dermatitis, such that the atopic dermatitis is treated in the subject. In some aspects, the methods comprise administering a 320-179 variant antibody, or composition thereof, to a subject in need of treatment for eczema, such that the eczema is treated in the subject. In some aspects, the methods comprise administering a 320-179 variant antibody, or composition thereof, to a subject in need of treatment for scleroderma, such that the scleroderma is treated in the subject.

The 320-179 variant antibodies described or exemplified herein may be used in the preparation of a medicament for use in the treatment of a respiratory tract disease. The 320-179 variant antibodies described or exemplified herein may be used in the preparation of a medicament for use in the treatment of a gastrointestinal disease. The 320-179 variant antibodies described or exemplified herein may be used in the preparation of a medicament for use in the treatment of arthritis. The 320-179 variant antibodies described or exemplified herein may be used in the preparation of a medicament for use in the treatment of a skin disease. The 320-179 variant antibodies described or exemplified herein may be used in the preparation of a medicament for use in the treatment of any one of asthma, COPD, pulmonary fibrosis, pulmonary sarcoidosis, allergic rhinitis, cystic fibrosis, inflammatory bowel disease, Crohn's disease, colitis, ulcerative colitis, irritable bowel syndrome, eosinophilic esophagitis, a gastrointestinal disease or condition associated with cystic fibrosis, arthritis, rheumatoid arthritis, atopic dermatitis, eczema, or scleroderma.

The 320-179 variant antibodies described or exemplified herein may be for use in the treatment of a respiratory tract disease. The 320-179 variant antibodies described or exemplified herein may be for use in the treatment of a gastrointestinal disease. The 320-179 variant antibodies described or exemplified herein may be for use in the treatment of asthma. The 320-179 variant antibodies described or exemplified herein may be for use in the treatment of a skin disease. The 320-179 variant antibodies described or exemplified herein may be for use in the treatment of COPD. The 320-179 variant antibodies described or exemplified herein may be for use in the treatment of pulmonary fibrosis. The 320-179 variant antibodies described or exemplified herein may be for use in the treatment of pulmonary sarcoidosis. The 320-179 variant antibodies described or exemplified herein may be for use in the treatment of allergic rhinitis. The 320-179 variant antibodies described or exemplified herein may be for use in the treatment of cystic fibrosis. The 320-179 variant antibodies described or exemplified herein may be for use in the treatment of inflammatory bowel disease. The 320-179 variant antibodies described or exemplified herein may be for use in the treatment of Crohn's disease. The 320-179 variant antibodies described or exemplified herein may be for use in the treatment of colitis. The 320-179 variant antibodies described or exemplified herein may be for use in the treatment of ulcerative colitis. The 320-179 variant antibodies described or exemplified herein may be for use in the treatment of eosinophilic esophagitis. The 320-179 variant antibodies described or exemplified herein may be for use in the treatment of a gastrointestinal disease or condition associated with cystic fibrosis. The 320-179 variant antibodies described or exemplified herein may be for use in the treatment of irritable bowel syndrome. The 320-179 variant antibodies described or exemplified herein may be for use in the treatment of rheumatoid arthritis. The 320-179 variant antibodies described or exemplified herein may be for use in the treatment of atopic dermatitis. The 320-179 variant antibodies described or exemplified herein may be for use in the treatment of eczema. The 320-179 variant antibodies described or exemplified herein may be for use in the treatment of scleroderma.

Also provided is an in vitro method for detecting TL1A in a tissue sample isolated from a subject, comprising contacting the antibody according to any one of claims 1-19 with a tissue sample isolated from a subject to form an antibody-TL1A complex, and detecting the complex in the tissue sample.

A 320-179 variant antibody may be used to detect TL1A-positive cells, for example, in a tissue sample obtained from a subject. The antibodies may be used to detect TL1A-positive peripheral blood mononuclear cells (PBMCs), for example, PBMCs obtained from a subject. The antibodies may be used to detect TL1A in the blood serum. Such methods may be carried out in vivo, ex vivo, in vitro, or in situ. In general, the methods comprise contacting any of the 320-179 variant antibodies described or exemplified herein with a tissue or cells, e.g., PBMCs, isolated from a subject to form an antibody-TL1A complex, and detecting the complex in the tissue or on the cells. The antibody may be labelled with a detectable label. The antibody may be detected with a secondary antibody that is labelled with a detectable label. The tissue may comprise or may be a biological fluid such as blood or blood serum. The tissue may comprise or may be respiratory tract tissue, such as lung tissue, sputum, bronchoalveolar lavage fluid, gastrointestinal tissue, or gastrointestinal lavage fluid. The tissue may comprise or may be skin or dermal tissue. The tissue may comprise or may be tissue of any joint in the body. The method may further comprise isolating the tissue from the subject. Such methods may be quantitative, for example, by quantifying the level of TL1A in the tissue, by quantifying the level of TL1A-positive cells, or by quantifying the level of TL1A on cells, or by quantifying the level of TL1A in the serum.

The disclosure also features kits comprising any of the 320-179 variant antibodies described and exemplified herein. The kits may be used to supply antibodies and other agents for use in diagnostic, basic research, or therapeutic methods, among others. In some aspects, the kits comprise any one or more of the 320-179 variant antibodies described or exemplified herein and instructions for using the one or more antibodies in a method for treating a respiratory tract disease, in a method for treating a gastrointestinal disease, or in a method for treating arthritis.

The following examples are provided to describe the disclosure in greater detail. They are intended to illustrate, not to limit, the disclosure.

### EXAMPLE 1

### Materials and Methods

Amino acid positions in these examples are numbered according to the Kabat numbering system. CDRs are defined according to the AbM method of CDR definition system throughout this document.

### 1.1. Generation of Variant Bundles.

The heavy- and light chain variable region amino acid sequences of antibody 320-179 (SEQ ID NOs:1 and 2 respectively) were used as templates for the design of point variants. 320-179 has been previously described in U.S. Publ. No. 2014/0255302 (VH is SEQ ID NO:186 and VL is SEQ ID NO:199 in that publication) as 320-179 (also described as C320-179). This antibody had favorable biophysical properties, was a potent inhibitor of TL1A and had a low predicted immunogenicity profile.

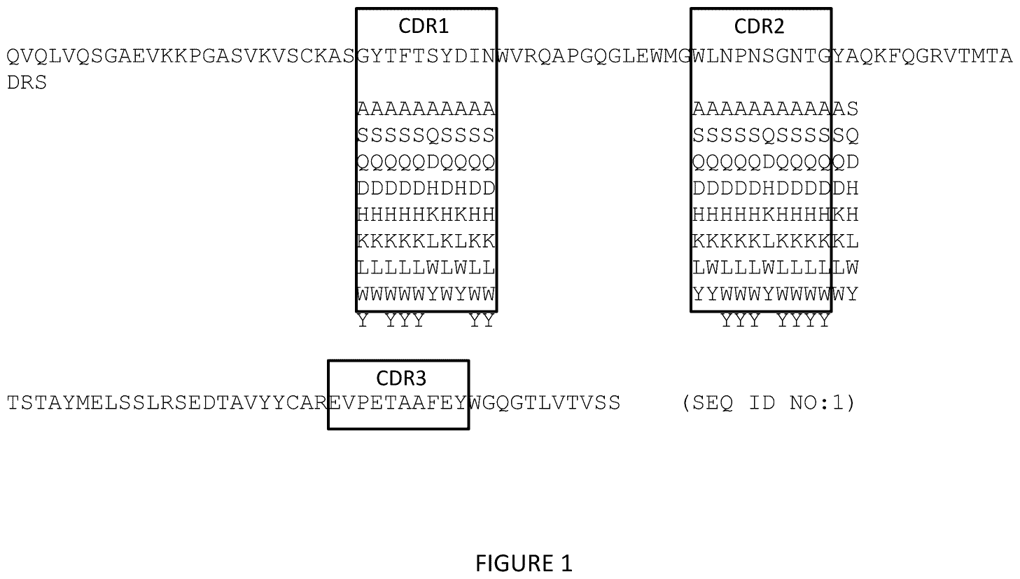

Antibody variants of 320-179 were made by substituting one of a group of nine representative amino acids—A, S, Q, D, H, K, L, W, Y—one at a time at one of each CDR amino acid position (as defined by AbM nomenclature) in the light chain CDR1 (CDR-L1), the light chain CDR3 (CDR-L3), the heavy chain CDR1 (CDR-H1) and the heavy chain CDR2 (CDR-H2). Antibody variants, incorporating A, S, Q, D, H, K, L, W, Y, were also made at position 59 and 60 in the variable heavy chain and at position 79 in the variable light chain. A complete list of all single substituted antibody variants generated is shown in FIGS. 1 (variable heavy chain) and 2 (variable light chain), respectively.

### 1.2. Construction of Vectors Expressing Antibodies.

Variable region variants were generated by back-translation of amino acid sequences into DNA sequences which were subsequently synthesized de novo by assembly of synthetic oligonucleotides. VH variants were subcloned into a mammalian expression vector containing a human constant region to produce full-length antibody heavy chains (human IgG1 heavy chain C_{H}1, hinge, C_{H}2 and C_{H}3 domains) (e.g., UniProt No. P01857). Similarly, V_{L} variants were subcloned into a mammalian expression vector containing a human lambda light chain constant region to produce full-length antibody lambda chains (SwissProt No. POCG05.1). In some instances, the full-length heavy chain and, separately, light chain, was back-translated into DNA sequences and subsequently synthesized de novo by assembly of synthetic oligonucleotides.

### 1.3. Expression of Antibody Variants.

Antibodies were produced by co-transfecting antibody heavy- and light chains into EXPI293® cells (Life Technologies, Carlsbad, CA). The day before transfection, the number of cells needed for the experiment was determined. For each 20 mL transfection, 3.6×10⁷ cells were required in 20 ml of EXPI293® Expression Medium. On the day prior to transfection, cells were seeded at a density of 0.9×10⁶ viable cells/mL and incubated overnight at 37° C. in a humidified atmosphere of 8% CO₂ in air on an orbital shaker rotating at 200 rpm. On the day of transfection, the cell number and viability were determined using an automated cell counter. Only cultures with >98% viable cells were used. For each 20 mL transfection, lipid-DNA complexes were prepared by diluting 10 ug of heavy chain DNA and 10 ug of light chain DNA in OPTI-MEM® (Life Technologies, Carlsbad, CA) I Reduced Serum Medium (Cat. no. 31985-062) to a total volume of 1.0 mL. 54 μL of EXPIFECTAMINE® 293 Reagent (Life Technologies, Carlsbad, CA) was diluted in OPTI-MEM® I medium to a total volume of 1.0 mL. Both vials were mixed gently and incubated for 5 minutes at room temperature. Following incubation, the diluted DNA was mixed with the diluted EXPIFECTAMINE® 293 Reagent and the DNA-EXPIFECTAMINE® 293 Reagent mixture and incubated a further 20 minutes at room temperature to allow the formation of DNA-EXPIFECTAMINE® 293 Reagent complexes. Following incubation, 2 mL of DNA-EXPIFECTAMINE® 293 Reagent complex was added to each 50mL bioreactor tube (TPP Techno Plastic Products AG). To the negative control tube, 2 mL of OPTI-MEM® (Life Technologies, Carlsbad, CA) I medium was added instead of DNA-EXPIFECTAMINE® 293 Reagent complex. The cells were incubated in a 37° C. incubator with a humidified atmosphere of 8% CO₂ in air on an orbital shaker rotating at 200 rpm. Approximately 16-18 hours post-transfection, 100 μL of EXPIFECTAMINE® 293 Transfection Enhancer 1 and 1.0 ml of EXPIFECTAMINER 293 Transfection Enhancer 2 were added to each bioreactor. Antibodies were harvested at approximately 72 hours post-transfection.

### 1.4. Purification of Antibody Variants.

Each antibody variant was expressed in EXPI293® cells in 20 ml of cell culture. Cultures were spun down in 50 mL falcon tubes at 3000×g for 20 minutes, and supernatants were filtered using a 0.22 μm filter (Corning). Supernatants were purified using a Gilson ASPEC GX274 robot. Briefly, SPE cartridges (Agilent, 12131014) packed with 1.2 mL MABSELECT SURE® protein A (GE Healthcare Bio-Sciences AB Uppsala, Sweden) resin were pre-equilibrated with 3 column volumes of 1×PBS. 18 mL of supernatant was run over the columns followed by a 4 ml 1×PBS wash. Each column was pre-eluted with 0.9 mL of 0.1 M citric acid, pH 2.9. Purified antibodies were eluted with 2 mL 0.1 M citric acid, pH 2.9. Antibodies were desalted into Sørensens PBS (59.5 mM KH₂PO₄, 7.3 mM Na₂HPO₄·2H₂O, 145.4 mM NaCl (pH˜5.8)) using PD-10 columns (GE Healthcare).

### 1.5. Antibody Expression and Antigen Binding as Determined by SPR.

Using a CM5 sensor chip (GE Healthcare) Protein A (Pierce) was coupled to the chip surface using an amine coupling kit (GE Healthcare). Protein A was coupled on flow cell 1 and 2 (or alternatively 3 and 4) using a BIACORE® T200. Supernatants from EXPI-293® cells containing antibody or alternatively purified antibodies (as described in 1.4) were passed over the surface of flow cell 2, while buffer (HBS-EP) was passed over flow cell 1. The amount of supernatant or purified protein (as well as the concentration) injected during the capture stage varied between runs and is specified in the header of the Tables 3-11. At the end of injection of the supernatant or purified antibody the change in response units was measured. This value was reported as Capture Level in Tables 3-11. To determine if the antibody bound TL1A, the TL1A was then passed over flow cells 1 and 2 and the response units measured prior to the end of the injection of TL1A (the association phase). This value is labeled as TL1A Binding Level (Early) in Tables 3-11. The response units were measured prior to the end of the dissociation phase. This value is labeled as TL1A Binding Level (Late) in Tables 3-11 and is a measure of the amount of antibody that has been lost from the surface of the chip as a result of dissociation of the TL1A—antibody complex. The sensorgrams were double referenced (flow cell 2 is subtracted from flow cell 1 and a buffer blank). As there were a large number of antibody variants to screen, these were screened across different runs (Tables 3-11). In each case (except Run 3) the parent antibody, 320-179 was included in the run, for comparison purposes. A summary of the conditions used in each run is below:

TABLE 1

| | | | Approx- | Conc. | | | |
|---|---|---|---|---|---|---|---|
| | | | imate | TL1A | TL1A Binding | TL1A Binding | |
| Run | Super- | | capture | sample | Leval (Early) | Level (Late) | |
| # | natant | Protein | leval (RU) | (ug/mL) | timepoint | timepoint | Notes |
| 1 | | x | 400 | 10 | 59 | 590 | Antibody diluted to 2 |
| | | | | | | | ug/mL, variable |
| | | | | | | | capture time |
| 2 | x | | 1000 | 10 | 44 | 220 | Supernatant diluted |
| | | | | | | | into running buffer, |
| | | | | | | | variable capture times |
| 2 | | x | 400 | 10 | 44 | 590 | Antibody diluted 2 |
| | | | | | | | ug/mL, variable |
| | | | | | | | capture time |
| 3 | x | | variable | 10 | 59 | 590 | Supernatant diluted |
| | | | | | | | into running buffer, 60 |
| | | | | | | | s capture |
| 4 | | x | 500 | 5 | 44 | 220 | Antibody diluted to 2 |
| | | | | | | | ug/mL into running |
| | | | | | | | buffer |
| 5 | x | | 100 | 5 | 44 | 220 | Supernatant diluted |
| | | | | | | | into running buffer, |
| 6 | | x | variable | 5 | 44 | 170 | Antibody diluted to 2 |
| | | | | | | | ug/mL into running |
| | | | | | | | buffer and captured |
| | | | | | | | for 45 s |
| 7* | | x | 400 | 5 | 44 | 220 | Antibody diluted to |
| | | | | | | | approx. 2 ug/mL |
| 8 | | x | 400 | 5 | 44 | 590 | Antibody diluted to 2 |
| | | | | | | | ug/mL, variable |
| | | | | | | | capture time |
| 9 | | x | 400 | 5 | 44 | 590 | Antibody diluted to 2 |
| | | | | | | | ug/mL, variable |
| | | | | | | | capture time |

*This run was performed on a Biacore ® A100

The binding of anti-TL1A to different species TL1A was also determined using SPR. The anti-TL1A antibody was captured on the surface of a Protein A. TL1A from either human, rat, mouse, rabbit, guinea pig, pig, dog, cat or cynomolgus monkey were flowed over the surface and the response units measured.

### 1.6. Production of TL1A.

Human TL1A was produced in the mammalian EXPI293® expression system, using a DNA expression construct coding for the extracellular domain (ECD) of human TL1A with an N-terminally located HIS and FLAG tag. Other species forms of TL1A were generated based on sequence listing on publically listed databases. These are summarized below:

TABLE 2

| Species TL1A | Public Database Reference | SEQ ID NO: |
|---|---|---|
| Human | UniProt: O95150 | 31 |
| Cynomolgus | SEQ ID NO: 125 from | 34 |
| Monkey | US Publ. No. 2014/0255302 | |
| Mouse | UniProt: Q5UBV8 | 35 |
| Rat | UniProt: Q8K3Y7 | 36 |
| Guinea Pig | UniProt: H0VFN8 | 37 |
| Cat | NCBI: XP_003995828.1 | 38 |
| Pig | UniProt: 13LL00 | 39 |
| Rabbit | UniProt: G1T1T1 | 40 |
| Dog | UniProt: J9P221 | 41 |

Culture supernatant containing the secreted TL1A protein was harvested by centrifugation at 2000×g for 10 mins to remove the cells. The TL1A protein was purified from the supernatant using a HISTRAP® HP column (GE Healthcare). The eluted protein was buffer-exchanged into PBS using a HILOAD® 16/60 Superdex 200 prep grade column (GE Healthcare) and ˜70 kDa fraction was separated by gel filtration on a HILOAD® 26/60 SUPERDEX® 200 prep grade column (GE Healthcare).

### 1.7. TF-1 Cell Line Potency Assay.

To determine which anti-TL1A antibodies functionally neutralize the biological activity of TL1A, antibodies were tested for their ability to neutralize TL1A-induced apoptosis in a TF-1 cell line. The TF-1 human erythroleukemic cell line (ATCC: CRL-2003) was maintained in culture under standard conditions. TF-1 cells (7.5×10⁴/well) were incubated in black-sided 96-well plates (Greiner) with human TL1A 100 ng/ml and cycloheximide 10 μg/ml to induce apoptosis. Test antibodies at a concentration of 10 μg/mL (66.7 nM) or less were added to the plates and incubated for 4 to 5 hours. Induction of apoptosis was then assessed using the Homogeneous Caspases Kit (Roche) according to manufacturer's instructions.

Data were normalized by expression as a percentage of maximum apoptosis (apoptosis levels achieved by human TL1A plus cycloheximide in the absence of anti-TL1A antibody).

### 1.8. Receptor Selectivity of Lead Antibodies.

TL1A binds both to its cognate signaling receptor, DR3, and to a decoy receptor, DcR3, which also serves as a decoy receptor for TNF family members Fas-L and LIGHT. Antibodies were assessed for their ability to inhibit binding of TL1A to its receptors in a competition ELISA. DR3/Fc Chimera (R&D Systems) or DcR3/Fc Chimera (R&D Systems) was coated onto a 96-well plate (Maxisorp, Nunc) at a concentration of 2 μg/ml. Serially diluted test antibodies were pre-incubated with single-site biotinylated human TL1A 1 μg/ml for 30 minutes then added to the DR3/Fc or DcR3/Fc coated wells. Bound TL1A was detected using streptavidin-horseradish peroxidase 1:2000 (BD Pharmingen). Data were normalized by expression as a percentage of maximum binding of TL1A to receptor in the absence of anti-TL1A antibody.

### 1.9. Kinetic Exclusion Assay.

This assay measures the free concentration of one of the binding partners without perturbing the equilibrium. Solutions can be prepared off-line, using unmodified proteins in solution, and affinity measurements can be read days after mixing to ensure that equilibrium has been reached. In a kinetic exclusion assay, one interactant (termed the constant binding partner, or CBP) is held at a constant concentration, while the other (termed the titrant) is serially diluted. Kinetic exclusion assays may be used to determine the dissociation constant (K_{D}) and affinity of an antibody-antigen interaction. In a typical kinetic exclusion assay, the titrant is immobilized to beads (e.g., Sepharose or PMMA beads) and is used to capture the CBP free in solution. A secondary labeled probe is then used to quantify the amount of captured CBP. The kinetic exclusion assay is reviewed in Darling, R K et al. (2004) ASSAY and Drug Development Technol. 2(6):647-57.

The components were combined and allowed to reach equilibrium. The kinetic exclusion assay was then used to measure the free fraction of the CBP. Equilibrium curves with multiple CBP concentrations were analyzed using the n-curve analysis tool within the KinExA® Pro software (Version 4.1.11, Sapidyne) to obtain robust K_{D} determinations. The interaction of 320-587 for human TL1A was examined using two orientations: (1) CBP is 320-587, titrant is TL1A, and (2) CBP is TL1A, titrant is 320-587.

### EXAMPLE 2

### Experimental Results

2.1. Selecting TL1A-Binding Variants with an Equivalent or Improved Off-Rate Relative to C320-179.

Variants of antibody 320-179 were constructed and expressed as described above. EXPI293® (Life Technologies Corp.) supernatants of each variant were assessed by BIACORE® (GE Healthcare) and the data obtained compared with that of the parental antibody 320-179. In some experiment the antibodies were purified using Protein A chromatography (See 1.4) and purified antibodies were used in BIACORE® (GE Healthcare) experiments. Tables 3-11 show the expression level of each variant, along with its binding to TL1A at an early and late time point. In later runs (Table 10 and 11) antibody variants containing more than one amino acid substitution were tested.

TABLE 3
SPR experiment - Run 1 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 410 | 140 | 117 |
| 320-184* | None | L79A | 414 | 141 | 118 |
| 320-185* | None | L79S | 401 | 137 | 114 |
| 320-186* | None | L79Q | 398 | 136 | 113 |
| 320-187* | None | L79D | 388 | 134 | 112 |
| 320-188* | None | L79H | 397 | 136 | 113 |
| 320-189* | None | L79K | 393 | 130 | 107 |
| 320-190* | None | L79W | 395 | 136 | 113 |
| 320-191* | None | L79Y | 408 | 140 | 117 |

*indicates antibodies that were selected for potency assay testing.

TABLE 4
SPR experiment-Run 2-Anti-TL1A antibodies binding to TL1A.

| | VH | VL | | TL1A | TL1A | | TL1A | TL1A |
|---|---|---|---|---|---|---|---|---|
| | Substitution | Substitution | | Binding | Binding | | Binding | Binding |
| | relative | relative | Capture | Level | Level | Capture | Level | Level |
| | to 320- | to 320- | Leval | (Early) | (Late) | Level | (Early) | (Late) |
| Antibody | 179 | 179 | Supernatant | | | Purified Antibody | | |
| 320-179 | None | None | 1133 | 287 | 261 | 410 | 140 | 117 |
| 320-192 | None | T24A | 960 | 238 | 218 | — | — | — |
| 320-193 | None | T24S | 990 | 249 | 227 | 409 | 103 | 91 |
| 320-194 | None | T24Q | 974 | 247 | 226 | 398 | 76 | 64 |
| 320-195 | None | T24D | 992 | 249 | 227 | — | — | — |
| 320-196 | None | T24H | 995 | 250 | 228 | — | — | — |
| 320-197 | None | T24K | 1017 | 255 | 232 | 401 | 105 | 94 |
| 320-198* | None | T24L | 1023 | 262 | 241 | — | — | — |
| 320-199 | None | T24W | 1050 | 251 | 233 | 404 | 97 | 85 |
| 320-200 | None | T24Y | 1027 | 251 | 231 | | | |
| 320-201* | None | S25A | 990 | 274 | 252 | 393 | 73 | 57 |
| 320-202* | None | S25Q | 1034 | 234 | 214 | — | — | — |
| 320-203 | None | S25D | 977 | 61 | 24 | 407 | 65 | 35 |
| 320-204 | None | S25H | 1003 | 193 | 155 | — | — | — |
| 320-205* | None | S25K | 1025 | 290 | 271 | 401 | 70 | 51 |
| 320-206 | None | S25L | 1000 | 257 | 235 | — | — | — |
| 320-207* | None | S25W | 1022 | 268 | 243 | — | — | — |
| 320-208 | None | S25Y | 1034 | 222 | 199 | — | — | — |
| 320-209* | None | S26A | 1007 | 222 | 200 | — | — | — |
| 320-210 | None | S26Q | 1033 | 225 | 202 | — | — | — |
| 320-211* | None | S26D | 1025 | 206 | 164 | 392 | 80 | 64 |
| 320-212 | None | S26H | 1013 | 226 | 205 | — | — | — |
| 320-213* | None | S26K | 1052 | 218 | 194 | 422 | 79 | 65 |
| 320-214 | None | S26L | 1038 | 221 | 196 | — | — | — |
| 320-215 | None | S26W | 1021 | 220 | 199 | — | — | — |
| 320-216 | None | S26Y | 1010 | 171 | 147 | — | — | — |
| 320-217 | None | S27A | 1076 | 258 | 235 | — | — | — |
| 320-218 | None | S27Q | 1081 | 239 | 218 | 412 | 92 | 80 |
| 320-219* | None | S27D | 1061 | 243 | 220 | — | — | — |
| 320-220 | None | S27H | 1049 | 253 | 230 | — | — | — |
| 320-221 | None | S27K | 1063 | 225 | 207 | 413 | 99 | 89 |
| 320-222 | None | S27L | 1037 | 156 | 96 | — | — | — |
| 320-223 | None | S27W | 1061 | 143 | 66 | — | — | — |
| 320-224 | None | S27Y | 1055 | 198 | 153 | — | — | — |
| 320-225 | None | S27aA | 1066 | 267 | 243 | — | — | — |
| 320-226* | None | S27aQ | 1035 | 250 | 228 | — | — | — |
| 320-227 | None | S27aD | 1022 | 242 | 218 | — | — | — |
| 320-228 | None | S27aH | 1072 | 259 | 235 | — | — | — |
| 320-229* | None | S27aK | 1057 | 268 | 251 | — | — | — |
| 320-230 | None | S27aL | 1087 | 245 | 223 | — | — | — |
| 320-231 | None | S27aW | 1078 | 271 | 243 | — | — | — |
| 320-232 | None | S27aY | 1051 | 261 | 235 | — | — | — |
| 320-233 | None | D27bA | 1118 | 255 | 235 | 409 | 48 | 16 |
| 320-234 | None | D27bS | 1089 | 262 | 239 | — | — | — |
| 320-235 | None | D27bQ | 1110 | 256 | 236 | — | — | — |
| 320-236 | None | D27bH | 1085 | 254 | 232 | 415 | 5 | 6 |
| 320-237 | None | D27bK | 1073 | 240 | 221 | 398 | 10 | 8 |
| 320-238 | None | D27bL | 1106 | 221 | 191 | — | — | — |
| 320-239 | None | D27bW | 1079 | 212 | 163 | — | — | — |
| 320-240 | None | D27bY | 1089 | 230 | 196 | — | — | — |
| 320-241* | None | I27cA | 1092 | 179 | 122 | — | — | — |
| 320-242 | None | 127cS | 1076 | 124 | 49 | — | — | — |
| 320-243 | None | I27cQ | 1065 | 71 | 26 | — | — | — |
| 320-244* | None | I27cD | 1083 | 21 | 20 | — | — | — |
| 320-245* | None | I27cH | 1084 | 59 | 26 | — | — | — |
| 320-246 | None | I27cK | 1089 | 32 | 23 | — | — | — |
| 320-247 | None | I27cL | 1078 | 205 | 162 | — | — | — |
| 320-248 | None | I27cW | 1110 | 96 | 30 | — | — | — |
| 320-249 | None | I27cY | 1096 | 77 | 28 | — | — | — |
| 320-250 | None | G28A | 1104 | 180 | 115 | — | — | — |
| 320-251 | None | G28S | 1085 | 134 | 50 | — | — | — |

*indicates antibodies that were selected for potency assay testing.

TABLE 5
SPR experiment - Run 3 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Supernatant | | |
| 320-252 | None | G28Q | 996 | 20 | 9 |
| 320-253 | None | G28D | 1073 | 6 | 6 |
| 320-254 | None | G28H | 1021 | 17 | 8 |
| 320-255 | None | G28K | 842 | 24 | 8 |
| 320-256 | None | G28L | 1055 | 30 | 10 |
| 320-257 | None | G28W | 981 | 83 | 18 |
| 320-258 | None | G28Y | 823 | 9 | 6 |
| 320-259 | None | A29S | 824 | 194 | 179 |
| 320-260 | None | A29Q | 836 | 182 | 165 |
| 320-261 | None | A29D | 956 | 86 | 29 |
| 320-262 | None | A29H | 891 | 209 | 190 |
| 320-263* | None | A29K | 848 | 121 | 123 |
| 320-264 | None | A29L | 831 | 199 | 184 |
| 320-265 | None | A29W | 1057 | 168 | 69 |
| 320-266 | None | A29Y | 934 | 218 | 192 |
| 320-267* | None | G30A | 1148 | 299 | 297 |
| 320-268 | None | G30S | 392 | 28 | 7 |
| 320-269 | None | G30Q | 764 | 126 | 93 |
| 320-270 | None | G30D | 511 | 9 | 5 |
| 320-271 | None | G30H | 1067 | 106 | 30 |
| 320-272 | None | G30K | 935 | 5 | 5 |
| 320-273 | None | G30L | 710 | 4 | 4 |
| 320-274 | None | G30W | 796 | 12 | 7 |
| 320-275 | None | G30Y | 751 | 44 | 10 |
| 320-276 | None | L31A | 1273 | 244 | 215 |
| 320-277* | None | L31S | 1741 | 387 | 361 |
| 320-278* | None | L31Q | 1696 | 389 | 374 |
| 320-279 | None | L31D | 561 | 144 | 128 |
| 320-280* | None | L31H | 881 | 228 | 211 |
| 320-281 | None | L31K | 321 | 22 | 6 |
| 320-282 | None | L31W | 811 | 75 | 16 |
| 320-283 | None | L31Y | 317 | 2 | 3 |
| 320-284 | None | G32A | 374 | 1 | 2 |
| 320-285 | None | G32S | 400 | 1 | 2 |
| 320-286 | None | G32Q | 576 | 1 | 4 |
| 320-287 | None | G32D | 339 | 0 | 2 |
| 320-288 | None | G32H | 422 | 1 | 3 |
| 320-289 | None | G32K | 463 | 1 | 3 |
| 320-290 | None | G32L | 361 | 1 | 2 |
| 320-291 | None | G32W | 414 | 2 | 3 |
| 320-292 | None | G32Y | 423 | 111 | 94 |
| 320-293 | None | V33A | 421 | 104 | 86 |
| 320-294 | None | V33S | 419 | 79 | 61 |
| 320-295 | None | V33Q | 414 | 109 | 94 |
| 320-296 | None | V33D | 428 | 118 | 104 |
| 320-297* | None | V33H | 420 | 95 | 77 |
| 320-298 | None | V33K | 416 | 114 | 100 |
| 320-299 | None | V33L | 420 | 51 | 13 |
| 320-300 | None | H34W | 417 | 111 | 95 |
| 320-301 | None | H34Y | 456 | 108 | 96 |
| 320-302* | None | H34A | 423 | 99 | 74 |
| 320-303 | None | H34S | 408 | 71 | 32 |
| 320-304 | None | H34Q | 401 | 103 | 82 |
| 320-305 | None | H34D | 424 | 34 | 10 |
| 320-306 | None | H34K | 452 | 147 | 133 |
| 320-307* | None | H34L | 873 | 216 | 200 |
| 320-308 | None | H34W | 458 | 106 | 73 |
| 320-309 | None | H34Y | 255 | 47 | 40 |
| 320-310 | None | Q89A | 348 | 70 | 62 |
| 320-311 | None | Q89S | 0 | 0 | 0 |

*indicates antibodies that were selected for potency assay testing.

TABLE 6
SPR experiment - Run 4 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 616 | 177 | 163 |
| 320-312 | None | Q89D | 529 | 10 | 10 |
| 320-313* | None | Q89H | 502 | 141 | 132 |
| 320-314* | None | Q89K | 598 | 165 | 158 |
| 320-315 | None | Q89L | 515 | 85 | 82 |
| 320-316 | None | Q89W | 535 | 65 | 62 |
| 320-317 | None | Q89Y | 570 | 86 | 83 |
| 320-318* | None | S90A | 578 | 160 | 149 |
| 320-319 | None | S90Q | 523 | 18 | 14 |
| 320-320 | None | S90D | 534 | 31 | 17 |
| 320-321 | None | S90H | 491 | 16 | 13 |
| 320-322 | None | S90K | 450 | 17 | 14 |
| 320-323 | None | S90W | 490 | 14 | 13 |
| 320-324 | None | S90Y | 528 | 13 | 12 |
| 320-325 | None | Y91A | 491 | 95 | 83 |
| 320-326* | None | Y91S | 550 | 126 | 120 |
| 320-327 | None | Y91Q | 493 | 130 | 119 |
| 320-328* | None | Y91H | 542 | 155 | 146 |
| 320-329 | None | Y91K | 600 | 9 | 11 |
| 320-330 | None | Y91L | 600 | 139 | 119 |
| 320-331* | None | Y91W | 615 | 173 | 176 |
| 320-332 | None | D92A | 531 | 66 | 44 |
| 320-333 | None | D92S | 559 | 95 | 67 |
| 320-334 | None | D92Q | 543 | 15 | 14 |
| 320-335 | None | D92H | 581 | 15 | 14 |
| 320-336 | None | D92K | 484 | 11 | 11 |
| 320-337 | None | D92L | 475 | 21 | 15 |
| 320-338 | None | D92W | 548 | 9 | 11 |
| 320-339 | None | D92Y | 509 | 10 | 11 |
| 320-340 | None | G93A | 548 | 145 | 132 |
| 320-341 | None | G93S | 560 | 149 | 135 |
| 320-342 | None | G93Q | 560 | 152 | 139 |
| 320-343 | None | G93D | 545 | 121 | 90 |
| 320-344 | None | G93H | 529 | 117 | 87 |
| 320-345* | None | G93K | 637 | 163 | 154 |
| 320-346 | None | G93L | 526 | 126 | 107 |
| 320-347 | None | G93W | 560 | 149 | 130 |
| 320-348 | None | G93Y | 541 | 144 | 127 |
| 320-349 | None | T94A | 556 | 112 | 88 |
| 320-350 | None | T94S | 502 | 130 | 118 |
| 320-351 | None | T94Q | 553 | 112 | 83 |
| 320-352 | None | T94D | 568 | 101 | 63 |
| 320-353 | None | T94H | 558 | 97 | 73 |
| 320-354 | None | T94K | 536 | 53 | 30 |
| 320-355 | None | T94L | 526 | 102 | 76 |
| 320-356 | None | T94W | 486 | 109 | 86 |
| 320-357 | None | T94Y | 563 | 119 | 91 |
| 320-358 | None | L95A | 501 | 82 | 50 |
| 320-359 | None | L95S | 503 | 57 | 27 |
| 320-360 | None | L95Q | 554 | 86 | 53 |
| 320-361* | None | L95D | 477 | 12 | 11 |
| 320-362 | None | L95H | 522 | 8 | 9 |
| 320-363 | None | L95Y | 566 | 17 | 16 |
| 320-364 | None | S95aA | 516 | 124 | 109 |
| 320-365 | None | S95aQ | 536 | 141 | 128 |
| 320-366 | None | S95aD | 487 | 62 | 31 |
| 320-367 | None | S95aH | 595 | 119 | 116 |
| 320-368 | None | S95aK | 602 | 50 | 26 |
| 320-369 | None | S95aL | 503 | 89 | 60 |
| 320-370 | None | S95aW | 570 | 66 | 39 |
| 320-371 | None | S95aY | 576 | 92 | 70 |

*indicates antibodies that were selected for potency assay testing.

TABLE 7
SPR experiment - Run 5 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Supernatant | | |
| 320-179 | None | None | 83 | 11 | 10 |
| 320-372 | None | A96S | 106 | 1 | 0 |
| 320-373 | None | A96Q | 99 | −1 | −1 |
| 320-374 | None | A96D | 98 | 1 | 0 |
| 320-375 | None | A96H | 108 | −2 | −1 |
| 320-376 | None | A96K | 98 | 3 | 1 |
| 320-377 | None | A96L | 101 | −1 | −1 |
| 320-378 | None | A96W | 105 | −1 | −1 |
| 320-379 | None | A96Y | 105 | 3 | 1 |
| 320-380 | None | L97A | 104 | 1 | 1 |
| 320-381 | None | L97S | 97 | 3 | 1 |
| 320-382 | None | L97Q | 106 | 0 | 0 |
| 320-383 | None | L97D | 102 | 3 | 1 |
| 320-384 | None | L97H | 98 | 3 | 2 |
| 320-385 | None | L97K | 108 | 1 | 1 |
| 320-386 | None | L97W | 104 | 1 | 0 |
| 320-387 | None | L97Y | 97 | 11 | 10 |
| 320-388 | G26A | None | 110 | 14 | 13 |
| 320-389 | G26S | None | 105 | 12 | 11 |
| 320-390 | G26Q | None | 100 | 12 | 11 |
| 320-391 | G26D | None | 106 | 14 | 13 |
| 320-392 | G26H | None | 109 | 12 | 11 |
| 320-393 | G26K | None | 100 | 12 | 11 |
| 320-394 | G26L | None | 106 | 13 | 12 |
| 320-395 | G26W | None | 104 | 13 | 12 |
| 320-396 | G26Y | None | 104 | 12 | 11 |
| 320-397 | Y27A | None | 109 | 12 | 12 |
| 320-398 | Y27S | None | 110 | 12 | 11 |
| 320-399 | Y27Q | None | 105 | 10 | 10 |
| 320-400 | Y27D | None | 106 | 11 | 11 |
| 320-401 | Y27H | None | 100 | 11 | 10 |
| 320-402 | Y27L | None | 98 | 11 | 10 |
| 320-403 | Y27W | None | 84 | 11 | 10 |
| 320-404 | T28A | None | 84 | 11 | 10 |
| 320-405 | T28S | None | 79 | 10 | 9 |
| 320-406 | T28Q | None | 82 | 8 | 7 |
| 320-407 | T28D | None | 88 | 12 | 11 |
| 320-408 | T28H | None | 81 | 13 | 12 |
| 320-409 | T28K | None | 83 | 10 | 10 |
| 320-410 | T28L | None | 86 | 11 | 10 |
| 320-411 | T28W | None | 82 | 10 | 9 |
| 320-412 | T28Y | None | 89 | 10 | 9 |
| 320-413 | F29A | None | 89 | 9 | 8 |
| 320-414 | F29S | None | 78 | 7 | 7 |
| 320-415 | F29Q | None | 85 | 5 | 4 |
| 320-416 | F29D | None | 81 | 9 | 8 |
| 320-417 | F29H | None | 85 | 9 | 8 |
| 320-418 | F29K | None | 85 | 10 | 9 |
| 320-419 | F29L | None | 83 | 11 | 9 |
| 320-420 | F29W | None | 85 | 11 | 10 |
| 320-421 | F28Y | None | 81 | 9 | 8 |
| 320-422 | T30A | None | 80 | 10 | 9 |
| 320-423 | T30S | None | 86 | 10 | 10 |
| 320-424 | T30Q | None | 86 | 11 | 10 |
| 320-425 | T30D | None | 89 | 14 | 12 |
| 320-426 | T30H | None | 84 | 10 | 9 |
| 320-427 | T30K | None | 88 | 11 | 10 |
| 320-428 | T30L | None | 88 | 11 | 10 |
| 320-429 | T30W | None | 90 | 12 | 11 |
| 320-430 | T30Y | None | 85 | 6 | 6 |
| 320-431 | S31A | None | 85 | 6 | 6 |

TABLE 8
SPR experiment - Run 6 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 449 | 111 | 102 |
| 320-432 | S31Q | None | 466 | 116 | 106 |
| 320-433 | S31D | None | 412 | 90 | 83 |
| 320-434 | S31K | None | 473 | 125 | 113 |
| 320-435 | S31L | None | 384 | 92 | 83 |
| 320-436 | S31W | None | 525 | 129 | 118 |
| 320-437 | S31Y | None | 501 | 119 | 109 |
| 320-438 | Y32A | None | 468 | 126 | 116 |
| 320-439 | Y32S | None | 464 | 119 | 108 |
| 320-440 | Y32Q | None | 408 | 108 | 97 |
| 320-441 | Y32D | None | 388 | 80 | 67 |
| 320-442 | Y32H | None | 490 | 124 | 113 |
| 320-443* | Y32K | None | 438 | 114 | 103 |
| 320-444 | Y32L | None | 458 | 107 | 95 |
| 320-445* | Y32W | None | 442 | 116 | 106 |
| 320-446 | D33A | None | 469 | 18 | 8 |
| 320-447 | D33S | None | 503 | 37 | 13 |
| 320-448 | D33Q | None | 482 | 7 | 6 |
| 320-449 | D33H | None | 483 | 76 | 59 |
| 320-450 | D33K | None | 536 | 1 | 3 |
| 320-451 | D33L | None | 497 | 19 | 8 |
| 320-452 | D33W | None | 445 | 84 | 78 |
| 320-453 | D33Y | None | 449 | 111 | 104 |
| 320-454 | I34A | None | 189 | 43 | 37 |
| 320-455 | I34S | None | 144 | 27 | 23 |
| 320-456 | I34Q | None | 214 | 52 | 46 |
| 320-457 | I34D | None | 55 | 6 | 4 |
| 320-458 | I34H | None | 239 | 59 | 53 |
| 320-459 | I34K | None | 93 | 20 | 17 |
| 320-460 | I34L | None | 441 | 114 | 105 |
| 320-461 | I34W | None | 465 | 87 | 82 |
| 320-462 | I34Y | None | 373 | 58 | 52 |
| 320-463* | N35A | None | 462 | 102 | 101 |
| 320-464* | N35S | None | 600 | 130 | 124 |
| 320-465 | N35Q | None | 476 | 92 | 78 |
| 320-466 | N35D | None | 360 | 93 | 86 |
| 320-467 | N35H | None | 350 | 44 | 24 |
| 320-468 | N35K | None | 200 | 2 | 2 |
| 320-469 | N35L | None | 315 | 69 | 61 |
| 320-470 | N35W | None | 329 | 2 | 3 |
| 320-471 | N35Y | None | 312 | 3 | 4 |
| 320-472 | None | S90L | 467 | 23 | 10 |
| 320-473 | None | Y91D | 453 | 69 | 55 |
| 320-474 | None | L95K | 596 | 2 | 4 |
| 320-475 | None | L95W | 727 | 8 | 8 |
| 320-476 | Y27K | None | 560 | 128 | 118 |
| 320-477* | S31H | None | 576 | 148 | 136 |
| 320-478 | A60L | None | 379 | 85 | 77 |
| 320-479 | A60W | None | 277 | 61 | 54 |
| 320-480 | A60Y | None | 319 | 79 | 73 |
| 320-483 | W50A | None | 492 | 4 | 6 |
| 320-484 | W50S | None | 555 | 3 | 6 |
| 320-485 | W50Q | None | 460 | 3 | 5 |
| 320-486 | W50D | None | 178 | 1 | 2 |
| 320-487 | W50H | None | 237 | 2 | 3 |
| 320-488 | W50K | None | 293 | 1 | 2 |
| 320-489 | W50L | None | 337 | 4 | 5 |
| 320-490 | W50Y | None | 393 | 14 | 8 |
| 320-491 | L51S | None | 356 | 85 | 76 |

*indicates antibodies that were selected for potency assay testing.

TABLE 9
SPR experiment - Run 7 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 301 | 61 | 56 |
| 320-492 | L51Q | None | 439 | 89 | 82 |
| 320-493 | L51D | None | 242 | 48 | 46 |
| 320-494 | L51H | None | 811 | 150 | 138 |
| 320-495 | L51K | None | 466 | 84 | 77 |
| 320-496 | L51W | None | 581 | 93 | 87 |
| 320-497 | N52A | None | 727 | 135 | 125 |
| 320-498 | N52S | None | 485 | 90 | 82 |
| 320-499 | N52Q | None | 551 | 122 | 113 |
| 320-500 | N52D | None | 472 | 69 | 53 |
| 320-501 | N52H | None | 533 | 118 | 108 |
| 320-502 | N52K | None | 659 | 88 | 59 |
| 320-503 | N52W | None | 484 | 41 | 6 |
| 320-504 | N52Y | None | 500 | 88 | 65 |
| 320-505 | P52aA | None | 570 | 118 | 110 |
| 320-506 | P52aS | None | 444 | 97 | 89 |
| 320-507 | P52aQ | None | 181 | 27 | 24 |
| 320-508 | P52aD | None | 203 | 19 | 12 |
| 320-509 | P52aH | None | 290 | 52 | 47 |
| 320-510 | P52aK | None | 289 | 44 | 33 |
| 320-511 | P52aL | None | 581 | 106 | 98 |
| 320-512 | P52aW | None | 746 | 126 | 118 |
| 320-513 | P52aY | None | 585 | 99 | 90 |
| 320-514 | N53A | None | 516 | 96 | 88 |
| 320-515 | N53S | None | 375 | 73 | 67 |
| 320-516 | N53Q | None | 461 | 92 | 86 |
| 320-517 | N53D | None | 493 | 55 | 50 |
| 320-518 | N53H | None | 882 | 169 | 153 |
| 320-519 | N53K | None | 993 | 217 | 196 |
| 320-520 | N53L | None | 1016 | 174 | 162 |
| 320-521 | N53W | None | 830 | 166 | 152 |
| 320-522 | N53Y | None | 693 | 141 | 129 |
| 320-523 | S54A | None | 476 | 88 | 82 |
| 320-524 | S54Q | None | 292 | 55 | 49 |
| 320-525 | S54D | None | 437 | 33 | 17 |
| 320-526 | S54H | None | 672 | 134 | 124 |
| 320-527 | S54K | None | 578 | 146 | 136 |
| 320-528 | S54L | None | 829 | 121 | 108 |
| 320-529 | S54W | None | 605 | 94 | 83 |
| 320-530 | S54Y | None | 425 | 77 | 67 |
| 320-531 | G55A | None | 331 | 66 | 61 |
| 320-532 | G55S | None | 648 | 10 | 7 |
| 320-533 | G55Q | None | 441 | 93 | 86 |
| 320-534 | G55D | None | 647 | 109 | 102 |
| 320-535 | G55H | None | 637 | 126 | 115 |
| 320-536 | G55K | None | 553 | 115 | 105 |
| 320-537 | G55L | None | 717 | 133 | 123 |
| 320-538 | G55W | None | 318 | 54 | 49 |
| 320-539 | N56A | None | 859 | 172 | 159 |
| 320-540 | N56S | None | 500 | 100 | 92 |
| 320-541 | N56Q | None | 504 | 94 | 84 |
| 320-542 | N56D | None | 839 | 97 | 86 |
| 320-543 | N56H | None | 597 | 138 | 126 |
| 320-544 | N56K | None | 690 | 120 | 112 |
| 320-545 | N56L | None | 518 | 109 | 100 |
| 320-546 | N56W | None | 341 | 67 | 61 |
| 320-547* | N56Y | None | 318 | 77 | 75 |
| 320-548 | T57A | None | 335 | 74 | 68 |
| 320-549 | T57S | None | 269 | 50 | 45 |
| 320-550 | T57Q | None | 635 | 112 | 103 |
| 320-551 | T57D | None | 280 | 44 | 36 |
| 320-552 | T57H | None | 595 | 104 | 97 |
| 320-553 | T57K | None | 494 | 84 | 78 |
| 320-554 | T57L | None | 506 | 66 | 51 |
| 320-555 | T57W | None | 674 | 62 | 30 |
| 320-556 | T57Y | None | 634 | 100 | 90 |
| 320-557 | G58A | None | 378 | 26 | 6 |
| 320-558 | G58S | None | 463 | 3 | 2 |
| 320-559 | G58Q | None | 535 | 9 | 5 |
| 320-560 | G58D | None | 907 | 36 | 9 |
| 320-561 | G58H | None | 539 | 14 | 6 |
| 320-562 | G58K | None | 326 | 2 | 1 |
| 320-563 | G58L | None | 258 | 2 | 2 |
| 320-564 | G58W | None | 345 | 72 | 67 |
| 320-565 | G58Y | None | 545 | 43 | 12 |
| 320-566 | Y59A | None | 720 | 120 | 110 |
| 320-567 | Y59S | None | 590 | 96 | 86 |
| 320-568 | Y59Q | None | 688 | 115 | 107 |
| 320-569 | Y59D | None | 408 | 79 | 73 |
| 320-570 | Y59H | None | 435 | 79 | 72 |
| 320-571 | Y59K | None | 394 | 78 | 72 |
| 320-572 | A60S | None | 459 | 98 | 90 |
| 320-573 | A60Q | None | 338 | 67 | 62 |
| 320-574 | A60D | None | 693 | 140 | 130 |
| 320-575 | A60H | None | 581 | 121 | 110 |
| 320-576 | A60K | None | 479 | 90 | 83 |
| 320-577 | L51A | None | 479 | 98 | 90 |
| 320-578 | L51Y | None | 415 | 83 | 77 |
| 320-579 | N52L | None | 214 | 46 | 41 |
| 320-580 | G55Y | None | 261 | 51 | 47 |
| 320-581 | Y59L | None | 337 | 70 | 65 |
| 320-582 | Y59W | None | 453 | 67 | 52 |

*indicates antibodies that were selected for potency assay testing.

TABLE 10
SPR experiment - Run 8 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 402 | 98 | 86 |
| 320-583* | None | G30A, Y91W | 404 | 108 | 103 |
| 320-584* | None | L31S, Y91W | 399 | 103 | 98 |
| 320-585* | None | L31Q, Y91W | 406 | 116 | 109 |
| 320-586* | None | H34L, Y91W | 405 | 108 | 103 |
| 320-587* | N56Y | Y91W | 402 | 123 | 122 |
| 320-588* | N56Y | G30A | 409 | 128 | 123 |
| 320-589* | N56Y | L31S | 399 | 120 | 109 |
| 320-590* | N56Y | L31Q | 405 | 130 | 121 |
| 320-591* | N56Y | H34L | 399 | 132 | 124 |
| 320-592* | N56Y | G30A, Y91W | 408 | 128 | 129 |
| 320-593* | N56Y | L31S, Y91W | 405 | 126 | 124 |
| 320-594* | N56Y | L31Q, Y91W | 407 | 134 | 132 |
| 320-595* | N56Y | H34L, Y91W | 403 | 129 | 128 |

*indicates antibodies that were selected for potency assay testing.

TABLE 11
SPR experiment - Run 9 - Anti-TL1A antibodies binding to TL1A.

| | | | | TL1A | TL1A |
|---|---|---|---|---|---|
| | VH | VL | | Binding | Binding |
| | Substitution | Substitution | Capture | Level | Level |
| | relative to | relative to | Level | (Early) | (Late) |
| Antibody | 320-179 | 320-179 | Purified Antibody | | |
| 320-179 | None | None | 402 | 108 | 98 |
| 320-596 | None | Y91F | 405 | 95 | 85 |
| 320-597 | None | L31Q, Y91F | 401 | 115 | 105 |
| 320-598 | None | H34L, Y91F | 403 | 112 | 101 |
| 320-599 | None | L31S, Y91F | 411 | 113 | 103 |
| 320-600 | None | G30A, Y91F | 399 | 111 | 104 |
| 320-601* | N56Y | Y91F | 409 | 121 | 120 |
| 320-602 | N56Y | L31Q, Y91F | 399 | 131 | 125 |
| 320-603 | N56Y | H34L, Y91F | 394 | 126 | 118 |
| 320-605 | N56Y | G30A, Y91F | 399 | 125 | 120 |
| 320-611* | N56Y | Y91W, G93K | 397 | 125 | 127 |
| 320-612 | None | Y91W, G93Y | 400 | 126 | 120 |
| 320-613* | N56Y | Y91W, G93Y | 408 | 135 | 131 |
| 320-614 | N56Y | G93K | 401 | 122 | 111 |
| 320-615 | N56Y | G93Y | 393 | 113 | 108 |
| 320-616 | N56Y | G93A | 396 | 120 | 112 |

*indicates antibodies that were selected for potency assay testing.

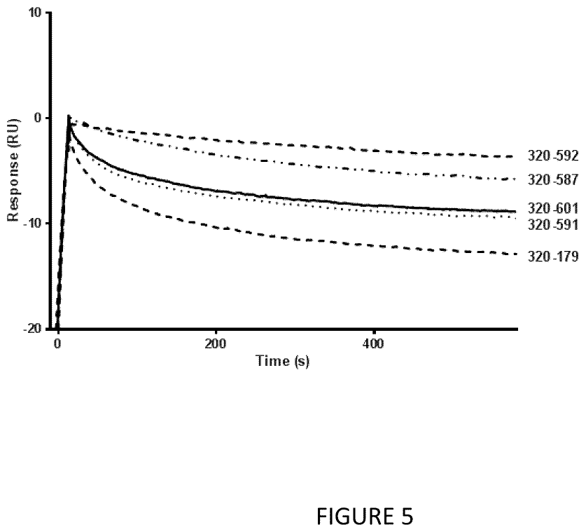

Antibodies that had capture levels similar or better than 320-179 as well as a TL1A Binding Level (Early) and TL1A Binding Level (Late) values that were in a similar range were taken forward into potency assays. These variants are indicated by * in Tables 3-11. A comparison of the off-rate as measured by SPR for several of the antibodies is shown in FIG. 5. Several of the antibodies dissociated at a slower rate than that of the parental antibody 320-179.

2.2 Anti-TL1A Antibodies with Improved Potency in Cell Based Assay.

To assess if improved off-rate correlated with an enhanced potency purified antibody, variants were run in the TL1A induced caspase activity assay in TF-1 cells. Potent antibodies act by binding to TL1A and inhibiting TL1A activation of the DR3 receptor. This receptor triggers an apoptosis pathway in which caspases are activated and can be detected using commercial reagents. In each experiment the antibody variant was compared to 320-179 for fold improvement in potency. The results are shown in Table 12.

TABLE 12
TL1A induced caspase potency assay in TF-1 cells: Inhibition by
anti-TL1A antibodies.

| | VH | VL | | | |
|---|---|---|---|---|---|
| | Substitution | Substitution | Antibody | 320-179 | Fold |
| | relative to | relative to | IC₅₀ | IC₅₀ | improve- |
| Antibody | 320-179 | 320-179 | (μg/mL) | (μg/mL) | ment |
| 320-184 | None | L79A | 0.010 | 0.030 | 3 |
| 320-185 | None | L79S | 0.020 | 0.030 | 2 |
| 320-186 | None | L79Q | 0.030 | 0.030 | 1 |
| 320-187 | None | L79D | 0.020 | 0.040 | 2 |
| 320-188 | None | L79H | 0.010 | 0.040 | 4 |
| 320-189 | None | L79K | 0.110 | 0.040 | 0 |
| 320-190 | None | L79W | 0.020 | 0.040 | 2 |
| 320-191 | None | L79Y | 0.020 | 0.040 | 2 |
| 320-198 | None | T24L | 0.010 | 0.010 | 1 |
| 320-201 | None | S25A | 0.005 | 0.010 | 2 |
| 320-202 | None | S25Q | 0.010 | 0.010 | 1 |
| 320-205 | None | S25K | 0.020 | 0.030 | 2 |
| 320-207 | None | S25W | 0.007 | 0.020 | 3 |
| 320-209 | None | S26A | 0.040 | 0.020 | 1 |
| 320-211 | None | S26D | 0.010 | 0.030 | 3 |
| 320-213 | None | S26K | 0.070 | 0.030 | 0 |
| 320-219 | None | S27D | 0.020 | 0.030 | 2 |
| 320-226 | None | S27aQ | 0.030 | 0.020 | 1 |
| 320-229 | None | S27aK | 0.040 | 0.060 | 2 |
| 320-241 | None | I27cA | 0.020 | 0.010 | 1 |
| 320-244 | None | I27cD | 5.030 | 0.010 | 0 |
| 320-245 | None | I27cH | 0.540 | 0.010 | 0 |
| 320-263 | None | A29K | 0.003 | 0.010 | 3 |
| 320-267 | None | G30A | 0.010 | 0.040 | 4 |
| 320-277 | None | L31S | 0.003 | 0.010 | 3 |
| 320-278 | None | L31Q | 0.010 | 0.020 | 2 |
| 320-280 | None | L31H | 0.006 | 0.020 | 3 |
| 320-297 | None | V33H | 0.005 | 0.020 | 4 |
| 320-302 | None | H34A | 0.010 | 0.020 | 2 |
| 320-307 | None | H34L | 0.010 | 0.040 | 4 |
| 320-313 | None | Q89H | 0.020 | 0.030 | 2 |
| 320-314 | None | Q89K | 0.020 | 0.030 | 2 |
| 320-318 | None | S90A | 0.020 | 0.050 | 3 |
| 320-326 | None | Y91S | 0.030 | 0.050 | 2 |
| 320-328 | None | Y91H | 0.020 | 0.020 | 1 |
| 320-331 | None | Y91W | 0.002 | 0.020 | 10 |
| 320-345 | None | G93K | 0.020 | 0.010 | 1 |
| 320-361 | None | L95D | 0.010 | 0.010 | 1 |
| 320-443 | Y32K | None | 0.060 | 0.060 | 1 |
| 320-445 | Y32W | None | 0.030 | 0.060 | 2 |
| 320-463 | N35A | None | 0.020 | 0.050 | 3 |
| 320-464 | N35S | None | 0.030 | 0.050 | 2 |
| 320-477 | S31H | None | 0.030 | 0.040 | 1 |
| 320-547 | N56Y | None | 0.004 | 0.040 | 10 |
| 320-583 | None | G30A, Y91W | 0.003 | 0.080 | 27 |
| 320-584 | None | L31S, Y91W | 0.008 | 0.080 | 10 |
| 320-585 | None | L31Q, Y91W | 0.003 | 0.040 | 13 |
| 320-586 | None | H34L, Y91W | 0.002 | 0.050 | 25 |
| 320-587 | N56Y | Y91W | 0.001 | 0.040 | 40 |
| 320-588 | N56Y | G30A | 0.004 | 0.020 | 5 |
| 320-589 | N56Y | L31S | 0.020 | 0.020 | 1 |
| 320-590 | N56Y | L31Q | 0.006 | 0.020 | 3 |
| 320-591 | N56Y | H34L | 0.002 | 0.020 | 10 |
| 320-592 | N56Y | G30A, Y91W | 0.003 | 0.020 | 7 |
| 320-593 | N56Y | L31S, Y91W | 0.004 | 0.060 | 15 |
| 320-594 | N56Y | L31Q, Y91W | 0.005 | 0.060 | 12 |
| 320-595 | N56Y | H34L, Y91W | 0.003 | 0.060 | 20 |
| 320-601 | N56Y | Y91F | 0.002 | 0.020 | 10 |
| 320-611 | N56Y | Y91W, G93K | 0.005 | 0.040 | 8 |
| 320-613 | N56Y | Y91W, G93Y | 0.010 | 0.040 | 4 |

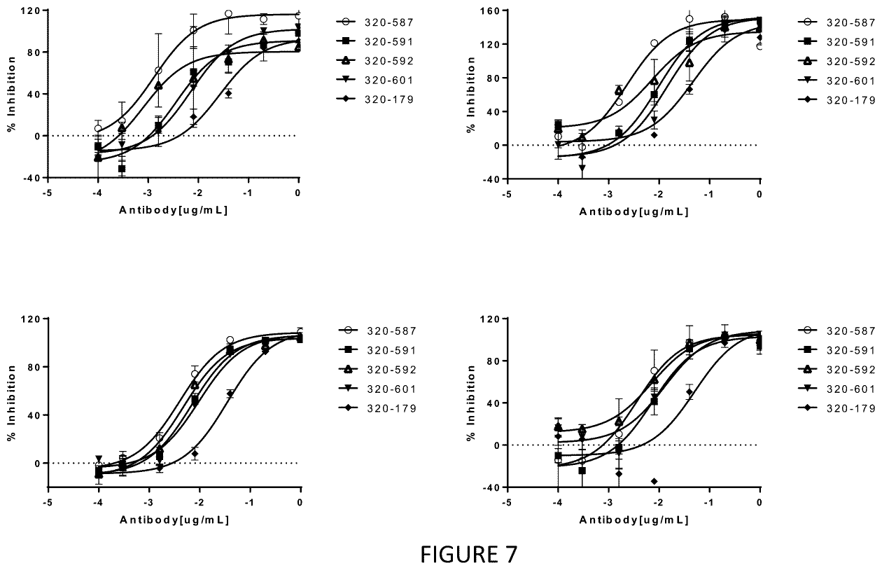

** Several of the antibodies with fold improvements greater than 10 fold improvement in in this potency were run in up to n = 7 assays. Results were consistent with the data shown table. FIG. 7 shows n = 4 replicates of several of the antibodies with improved potency compared to 320-179.

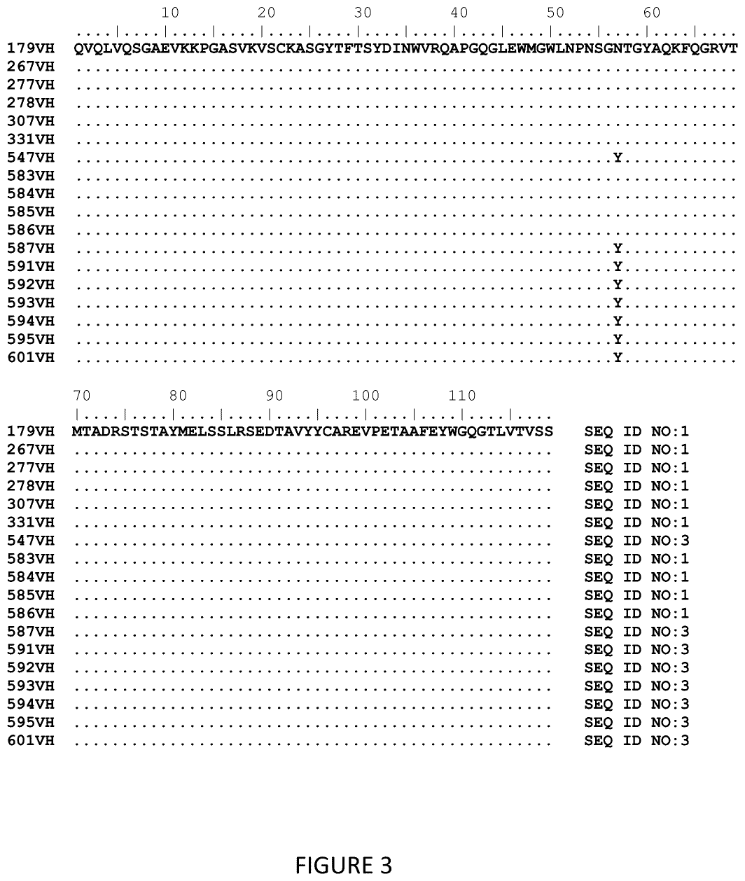

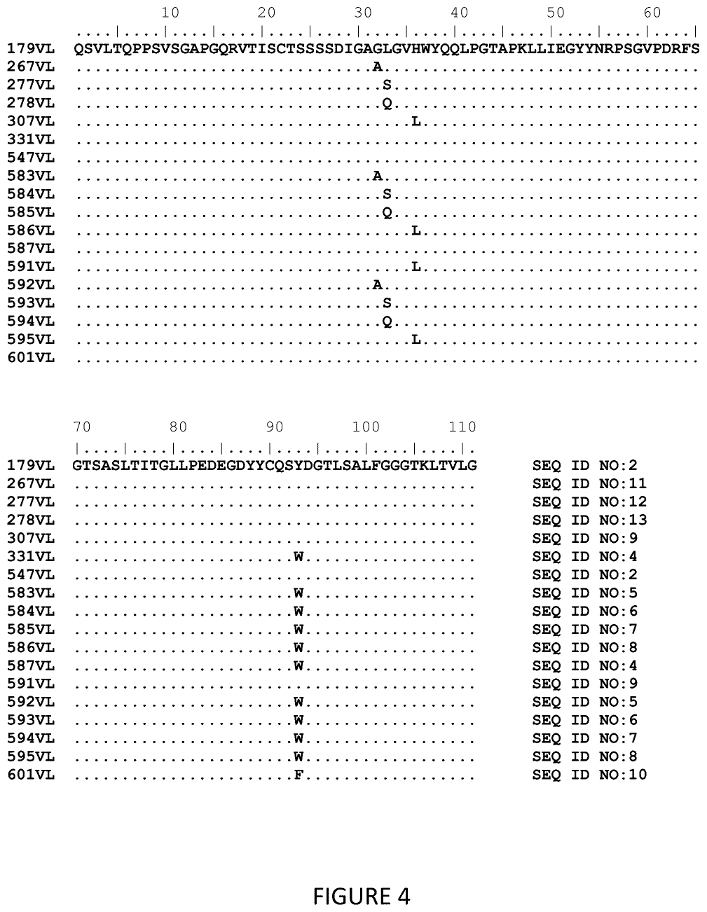

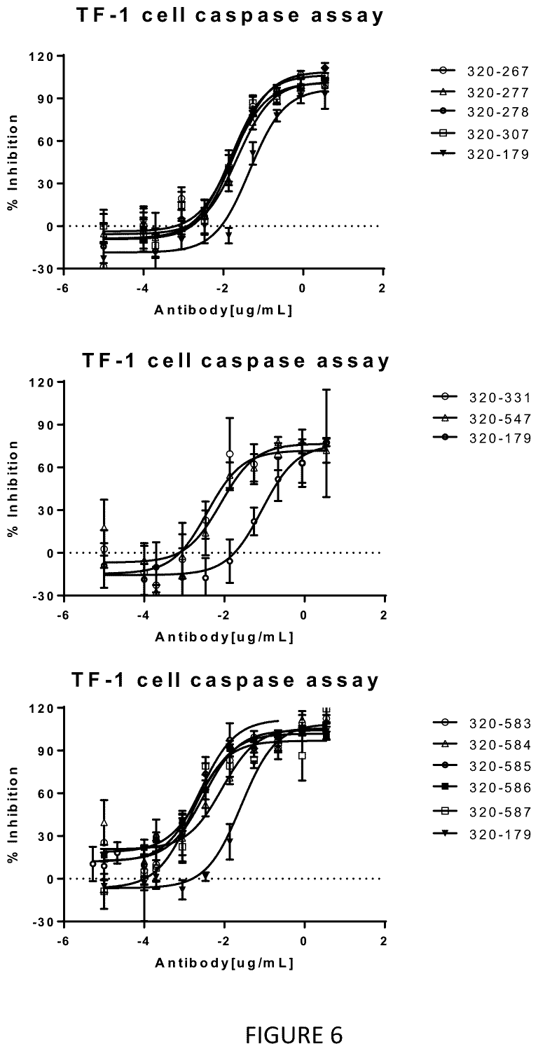

As demonstrated in Table 12 and in FIG. 6, several of the single substitution antibodies tested had superior potency compared to 320-179. Of all the single substitution antibody variants tested two had greater than 10 fold improvement in potency when compared to 320-179. These variants were 320-331 (which contained a Y91W substitution in the variable light chain) and 320-547 (which contained a N56Y substitution in the variable heavy chain) (FIG. 6). This result is unexpected, as typically CDR3 of the antibody VH is dominantly involved in antibody binding, while in contrast, the N56Y substitution that was identified has substantial influence on binding lies in CDR2 of the antibody VH. When variants were made incorporating either Y91W from the VL or separately N56Y in the VH with other substitutions that improved the potency, highly potent antibodies were obtained. When the Y91W VL substitution was combined with the N56Y VH substitution into one antibody, 320-587, the fold improvement in potency compared to 320-179 was 40. FIG. 7 shows four different repeat experiments demonstrating the potency increase of four antibodies 320-587, 320-591, 320-592, and 320-601 compared to 320-179. The sequences of these antibodies with improved potency compared to 320-179 are shown in FIG. 3 (variable heavy chain) and FIG. 4 (variable light chain).

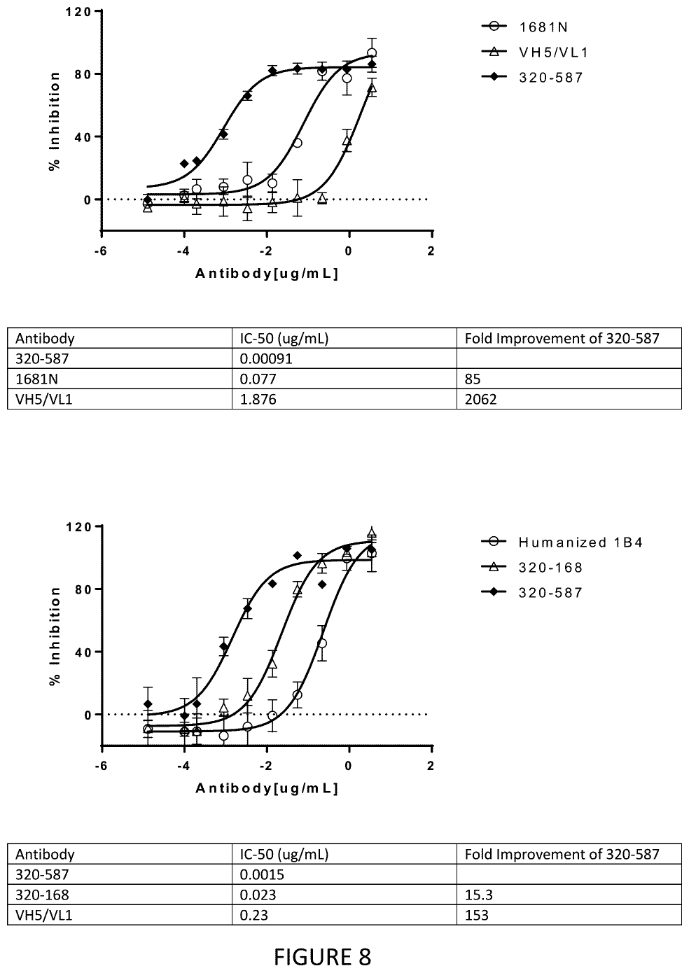

A comparison of the potency of 320-587 compared to other previously described anti-TL1A antibodies was performed. These previously described antibodies include antibody 1681N described in U.S. Pat. No. 8,642,741 (VH is SEQ ID NO:18; VL is SEQ ID NO. 26), antibody VH5/VL1 from U.S. Publ. No. 2014/0308271 (VH is SEQ ID NO:24; VL is SEQ ID NO:17), humanized 1B4 as described in U.S. Pat. No. 8,263,743 (VH is SEQ ID NO:74; VL is SEQ ID NO:75) and 320-168 (also called C320-168) as described in U.S. Publ. No. 2014/0255302A1 (VH is SEQ ID NO:181; VL is SEQ ID NO:194). FIG. 8 shows that 320-587 has superior potency in the cell based assay compared to these previously described antibodies, making it the most potent anti-TL1A antibody described.

2.3. DR3 and DcR3 Receptor Competition Assays.

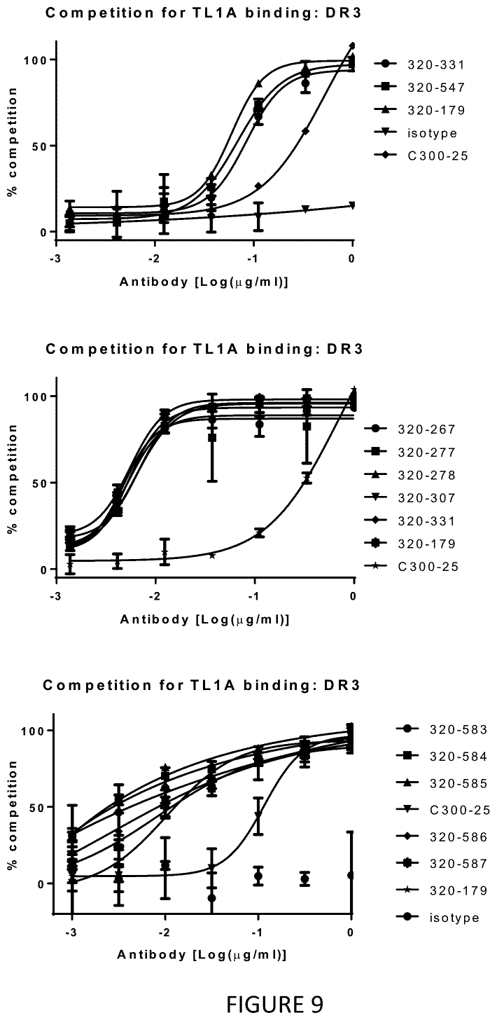

Antibodies that displayed increased potency compared to 320-179 were screened for their ability to inhibit TL1A binding to its cognate signalling receptor, DR3, or a decoy receptor, DcR3. All anti-TL1A antibodies tested showed inhibition of TL1A binding to DR3, when compared to an isotype control (FIG. 9). This confirms that the antibodies inhibit TL1A activity by blocking the TL1A-DR3 interaction.

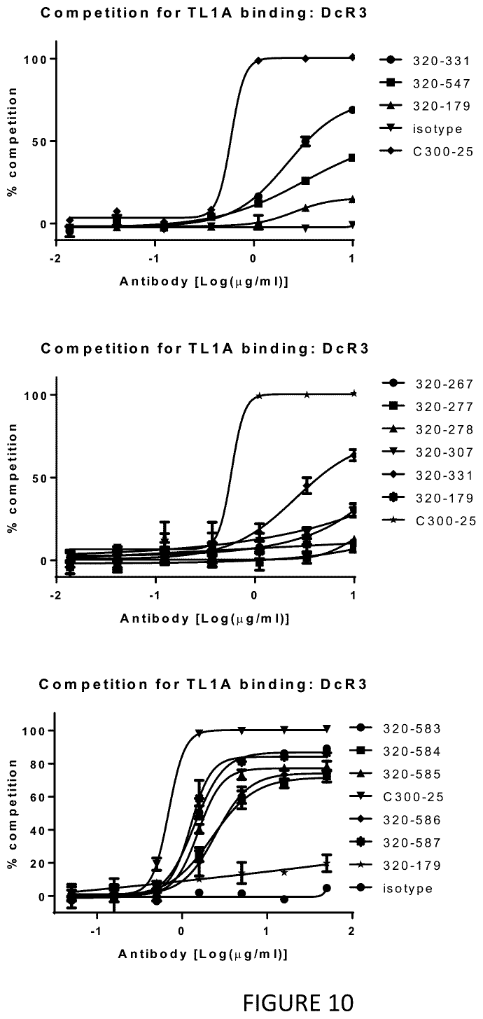

In previous experiments described in U.S. Publ. No. 2014/0255302A1 (Example 4), antibody 320-179 (320-179) was tested in receptor competition assay and shown to selectively inhibit the binding of TL1A to DR3 but not to DcR3 (FIG. 10). In experiments presented herein in FIG. 10 it is again shown that 320-179 does not inhibit the TL1A-DcR3 interaction. This stands in contrast with the improved anti-TL1A antibodies tested. Antibodies with improved potency in the TF-1 cell assay (as described in Section 2.2), including antibody 320-587, consistently inhibited the TL1A-DcR3 interaction.

In summary, the parental antibody 320-179 was capable of inhibiting the TL1A-DR3 interaction but not the TL1A-DcR3 interaction. Several antibodies with improved potency such as 320-267 (VH is SEQ ID No:1; VL is SEQ ID No:11), 320-277 (VH is SEQ ID NO:1, VL is SEQ ID NO 12), 320-278 (VH is SEQ ID NO 1; VL is SEQ ID NO 13) and 320-591 (VH is SEQ ID NO:3, VL is SEQ ID NO 9) inhibited the TL1a-DR3 interaction but not the TL1A-DcR3 interaction. Several antibodies with improved potency such as 320-331, 320-547, 320-583, 320-584, 320-585, 320-586, 320-587 and variants of these antibodies, are capable of inhibiting both the TL1A-DR3 and the TL1A-DcR3 interaction.

2.4. Species Cross-Reactivity of 320-587 was Tested for its Ability to Bind to Recombinantly Produced TL1A from Different Species.

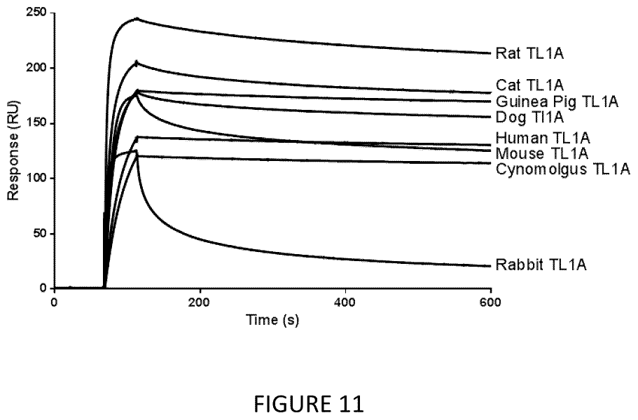

The antibody bound to TL1A from all species tested (FIG. 11). The binding of 320-587 to human, rat, guinea pig, dog, cat, cynomolgus monkey TL1A had a slow dissociation rate, indicating a high affinity interaction. The antibody bound mouse and rabbit TL1A and had a fast dissociation rate.

### 2.5. Pre-Clinical Efficacy Models for Testing Anti-TL1A Antibodies in the Following Animal Models of Disease:

Asthma: allergen-induced asthma—rodent (mouse, rat or guinea pig) is sensitized by intradermal injection of ovalbumin (OVA), especially OVA derived from chicken eggs, plus alum and then challenged at least 2 weeks later by aerosolized of nebulized OVA, causing asthma-like symptoms including airways hyerreactivity, influx of eosinophils and increased production of cytokines (e.g., Hylkema et al., 2002, Clin. Exp. Immunol. 129:390-96). Such a model could be modified by repeated challenge to present a more chronic disease profile with increased airways remodeling and fibrosis induction (e.g., Bos et al., 2007, Eur. Respir. J. 30:653-661). Alternative allergens, such as house dust mite, may also be used (e.g., Lambert et al., 1998, Am. J. Respir. Crit. Care Med. 157:1991-9). Alternatively, a nonhuman primate (e.g., cynomolgus macaque) may be sensitized and challenged with an environmental antigen such as Ascaris suum, leading to airways hyerreactivity, influx of eosinophils and increased production of cytokines (e.g., Wegner et al., 1991, J. Allergy Clin. Immunol.87:835-41).

COPD: Smoke inhalation-induced airways inflammation—rodent (mouse, rat or guinea pig) will be exposed to cigarette smoke 3-7 times a week for at least 4 weeks causing a pulmonary disease similar to COPD, characterized by lung accumulation of neutrophils, increased inflammatory cytokine production, lung fibrosis and pulmonary hypertension (e.g., Davis et al. (2012) PLoS One 7:e33304). A more severe form of disease may be induced by including repeat bacterial or viral infection into the lungs during smoke exposure (e.g., Li et al. (2012) Biol. Pharm. Bull. 35:1752-60). Rodents with smoke-induced COPD will be treated with anti-TL1A antibodies and screened for treatment efficacy.

Pulmonary fibrosis: Bleomycin-induced pulmonary fibrosis—rodent (mouse, rat or guinea pig) will be treated with bleomycin either by intratracheal/intranasal instillation or intravenous injection once or twice weekly for at least 3 weeks. This treatment induces significant and stable pulmonary fibrosis (e.g., Pinart et al. (2009) Resp. Physiol. Neurobiol. 166:41-46). Rodents with bleomycin-induced pulmonary fibrosis will be treated with anti-TL1A antibodies and screened for treatment efficacy.

Cystic Fibrosis: CFTR knockout ferret model—ferrets homozygous for gene knockout, or known disease-related mutations, of CFTR (causative gene in cystic fibrosis) spontaneously develop a cystic fibrosis-like disease characterized by mucus obstruction of airways, atelectasis, interstitial pneumonia and repeated lung infections with progressive lung bacterial colonization (e.g., Sun et al. (2014) Am. J. Respir. Cell Mol. Biol. 50:502-12). CFTR^{-/-} ferrets will be treated with anti-TL1a antibodies and screened for treatment efficacy.

Irritable Bowel Syndrome: Stress-induced visceral hypersensitivity—Stress will be induced in rats by either neonatal-maternal separation (e.g., Coutinho et al. (2002) Am. J. Physiol. Gastrointest. Liver Physiol. 282:G307-16) or restraint of adults (e.g., Shen et al. (2010) J. Neurogastroenterol. Motil. 16:281-90). This is expected to produce altered colonic motility and visceral hypersensitivity similar to that observed in IBS patients. Stressed rats will be treated with anti-TL1A antibodies and screened for treatment efficacy.

Rheumatoid Arthritis: Collagen-induced arthritis—rodent (mouse, rat or guinea pig) will be immunized and boosted with collagen in adjuvant. Animals develop bilateral foot swelling and erythema, inflammatory infiltrate into joint area and joint damage (e.g., Bendele et al. (1999) Toxicol. Pathol. 27:134-42). Rodents with collagen-induced arthritis will be treated with anti-TL1A antibodies and screened for treatment efficacy.

Eosinophilic esophagitis: Intranasal Aspergillus fumigatus-induced eosinophilic esophagitis. Mice exposed to repeat intranasal instillation of A. fumigatis develop marked esophageal eosinophilia, epithelial dysplasia and hyperplasia, and free eosinophil granules (e.g., Mishra et al. (2001) J. Clin. Invest. 107:83-90). Similarly, repeat aerosol exposure to ovalbumin over a two week period in sensitized guinea pigs causes esophageal eosinophilia with infiltration of both eosinophils and mast cells into the epithelial layer (e.g., Liu et al. (2015) Am. J. Physiol. Gastrointest. Liver Physiol. 308:G482-488). Mice or guinea pigs with eosinophilic esophagitis will be treated with anti-TL1A antibodies and screened for treatment efficacy.

### 2.6. Use of TL1A Antibodies in Detecting Samples Containing TL1A Antibodies of the Disclosure can be Used to Detect TL1A in Human Samples.

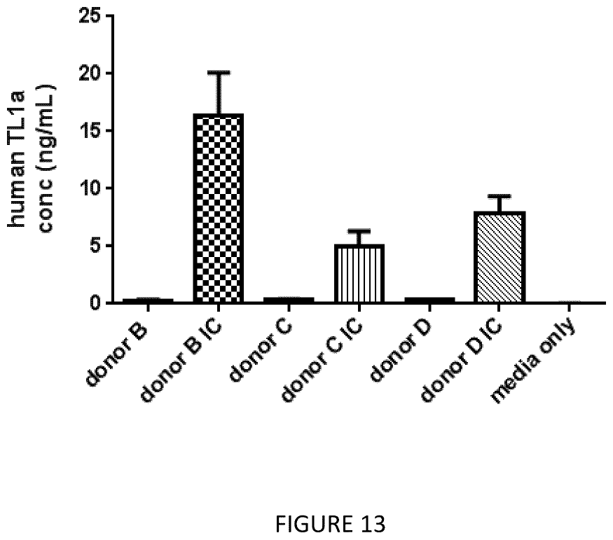

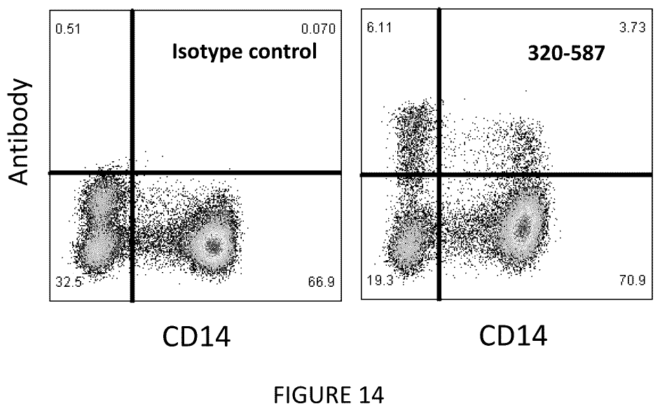

320-587 was used to detect human TL1A secreted from human PBMCs stimulated with immune complexes (FIG. 13) in ELISA format. 320-587 was also used to detect a population of human PBMCs that express membrane TL1A on their surface in flow cytometry experiments (FIG. 14).

### 2.7. Affinity Measurements of Anti-TL1A Antibody Binding to Human TL1A by Kinetic Exclusion Assay.

Time to reach equilibrium with 320-587 as CBP: First, the Kₒₙ rate for the 320-587/TL1A interaction was measured. Briefly, a solution was prepared by mixing 320-587 and TL1A, and aliquots were removed at various time points over 3 hours. Free 320-587 was captured by passing the solution over a column packed with Sepharose beads coated with 20 μg/mL TL1A. Captured 320-587 was detected with an Alexa Fluor® 647-conjugated anti-human antibody (0.5 μg/mL). This assay was repeated two times yielding Kₒₙ rates of 8.35×10⁵ Ms⁻¹ and 7.45×10⁵ Ms⁻¹, with an average Kₒₙ rate of 7.90×10⁵ Ms⁻¹. The Kₒₙ rate was then used to estimate the amount of time required to reach equilibrium at various concentrations of 320-587 using the theoretical binding curve tool provided on the Sapidyne website.

K_{D} determination with 320-587 as CBP. The CBP, 320-587, was diluted in assay buffer (DPBS supplemented with 1 mg/ml BSA) to final concentrations of 15, 50 and 150 pM. The titrant, human TL1A was diluted in assay buffer to create a concentration series of 0.1, 0.3, 1, 3, 10, 30, 100, 300, 1000 and 3000 pM. Using the time to reach equilibrium determined above, curves that contained either 50 or 150 pM 320-587 were allowed to come to equilibrium in a 25° C. incubator for 2 days. Curves that contained 15 pM 320-587 were allowed to come to equilibrium in a 25° C. incubator for 3 days. Following the equilibration period, the free fraction of 320-587 in each reaction was quantitated as described in above. The K_{D} values were determined using n-curve analysis of equilibrium curves generated with 15, 50 and 150 pM 320-587.

Time to reach equilibrium with TL1A as CBP: The time to reach equilibrium in this orientation was estimated using the Kₒₙ for the 320-587/TL1A interaction, determined as described in 2.8 above. In this format, the free fraction of TL1A was captured by passing the solution over a column packed with PMMA beads coated with 30 μg/ml 320-587. Captured TL1A was detected with an anti 6x-his DyLight 650 antibody (0.75 μg/mL). This assay was repeated two times yielding Kₒₙ rates of 6.11×10⁵ Ms⁻¹ and 5.74×10⁵ Ms⁻¹, with an average Kon rate of 5.93×10⁵ Ms⁻¹.

K_{D} determination with TL1A as CBP: The CBP, human TL1A, was diluted in assay buffer to final concentrations of 30, 100 and 300 pM. The titrant, 320-587, was diluted in assay buffer to create a concentration series of 0.05, 0.15, 0.5, 1.5, 5, 15, 50, 150, 500, 1500 and 5000 pM.

Using the time to reach equilibrium determined above, all curves were allowed to come to equilibrium in a 25° C. incubator for 3 days. Following the equilibration period, the free fraction of TL1A in each reaction was quantitated as described above. The K_{D} values were determined using n-curve analysis of equilibrium curves generated with 30, 100 and 300 pM TL1A.

Good agreement was observed between the two KinExA methods as well as a relatively low % error. The K_{D} value for the interaction of TL1A with 320-587 determined using 320-587 as the CBP was 40.97±8.33 PM (Table 13), while the K_{D} obtained using TL1A as the CBP was 41.52±13.5 pM (Table 14).

TABLE 13
Affinity: Sepharose beads coated with TL1A; 320-587 as the CBP.

| Assay No: | K_{D} (pM) | % Error |
|---|---|---|
| 1 | 40.43 | 4.57 |
| 2 | 52.03 | 5.61 |
| 3 | 46.31 | 4.76 |
| 4 | 32.42 | 3.74 |
| 5 | 33.66 | 3.9 |
| Average | 40.97 | |
| SD | 8.33 | |
| % CV | 20 | |

TABLE 14
Affinity: PMMA beads coated with 320-587; TL1A as the CBP.

| Assay No: | K_{D} (pM) | % Error |
|---|---|---|
| 1 | 33.39 | 5.33 |
| 2 | 57.1 | 5.51 |
| 3 | 34.07 | 3.9 |
| Average | 41.52 | |
| SD | 13.5 | |
| % CV | 33 | |

### 3.0. Preclinical Efficacy Models for Testing Anti-TL1a Antibodies.

### 3.0.1. Asthma.

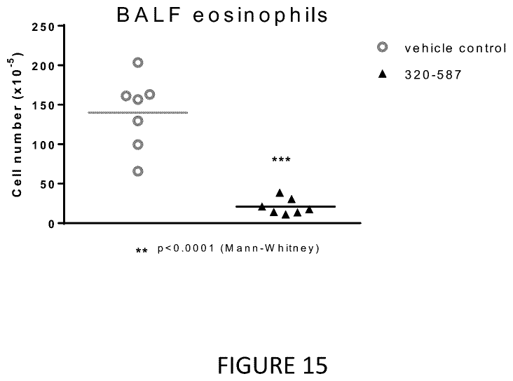

Acute ovalbumin-induced asthma in rats. Brown-Norway rats were sensitized with OVA by i.p. injection on day 0 then challenged with OVA aerosol daily on days 35-42. Rats were treated with antibody 320-587 or vehicle by i.v. injection on days 14, 21, 28 and 35. Bronchoalveolar lavage fluid (BALF) was assessed for total and differential cells on day 43. Treatment was found to significantly reduce BALF eosinophils (FIG. 15).

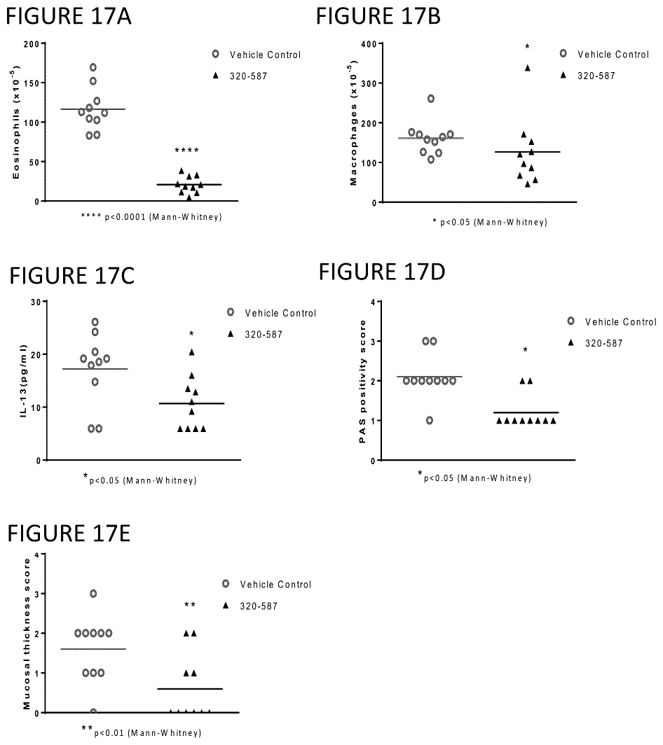

Chronic ovalbumin-induced asthma in rats. Rats were sensitized with OVA plus alum by i.p. injection on days 0 and 7, and then challenged with OVA aerosol twice weekly for 3 weeks starting on day 14 through day 31, and on 5 consecutive days from days 37 to 42. Animals were treated with antibody 320-587 or vehicle i.v. on days 24, 29, 34 and 39. BALF was assessed for total and differential cells, and a panel of cytokines on day 43. Lung sections were stained with hematoxylin and eosin (H&E), and periodic-acid Schiff (PAS), and assessed for a range of pathologies. Treatment with 320-587 significantly decreased BALF eosinophils and macrophages (FIGS. 17A and 17B), BALF IL-4 and IL-13 (FIG. 17C), goblet cell hyperplasia (FIG. 17D) and the thickness of the bronchial epithelial layer (FIG. 17E), compared to the vehicle.

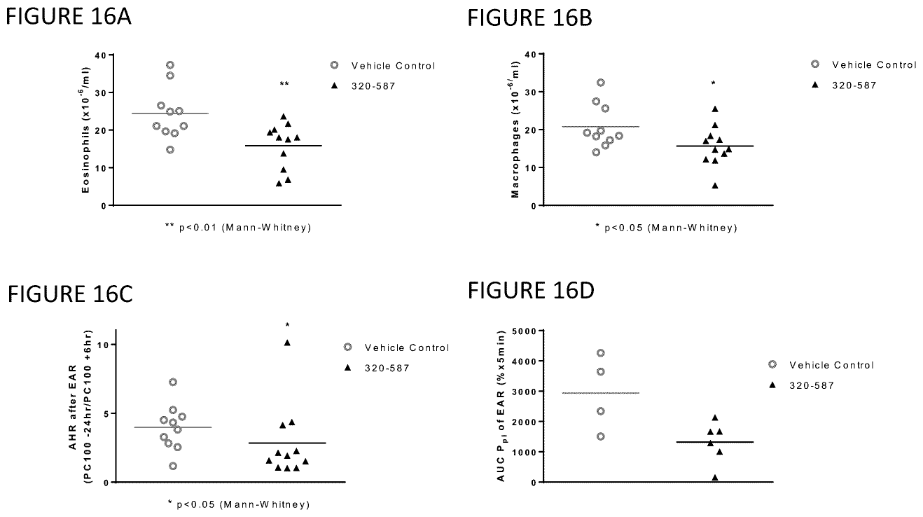

Acute ovalbumin-induced asthma in guinea pigs. Male Dunkin Hartley guinea pigs were sensitized to ovalbumin and thereafter underwent surgery to install a balloon catheter to measure lung function and early and late asthmatic reactions. On day 16, 20, 24 and 28, animals were treated i.p. with antibody 320-587 or vehicle. Challenge with ovalbumin (0.05-0.1%) aerosol was performed 30 minutes after the last treatment. Airway responsiveness (AHR) to histamine was measured 24 h before challenge, 6 h after challenge (directly after the early asthmatic reaction) and 24 h after challenge (directly after the late asthmatic reaction). The nature and size of the early and late asthmatic reactions was also be recorded by online registration of lung function over the entire 24 h period. Animals were sacrificed 25 h after challenge and bronchoalveolar lavage performed. BALF was assessed for total and differential cells. Treatment with 320-587 significantly decreased both eosinophils and macrophages in BALF (FIGS. 16A and 16B) as well as ameliorating AHR after the early asthmatic reaction (FIG. 16C) and the overall magnitude of the early asthmatic reaction (FIG. 16D), as compared to vehicle.

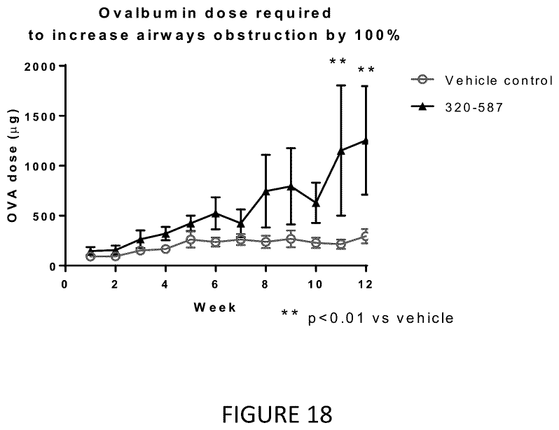

Chronic ovalbumin-induced asthma in guinea pigs. Male Dunkin Hartley guinea pigs were sensitized to ovalbumin, and 4 weeks thereafter challenged with ovalbumin weekly for 12 weeks. Ovalbumin challenge (0.05-0.5%) was performed by inhalation of aerosolized solution until airway obstruction was observed. Animals were treated with antibody 320-587 or vehicle i.p. every 5 days starting week 8 of ovalbumin challenges. Airways function, by means of airways responsiveness to histamine, was measured before the initial challenge, 24 hours before the final challenge, and 6 hours after the final challenge. Although no effect on AHR induced by histamine challenge was observed, antibody 320-587 significantly decreased the allergic response to OVA, as progressively increasing doses of OVA were required to induce airways obstruction (FIG. 18).

The differences in antibody therapeutic effect observed in the acute and chronic asthma models in guinea pigs is believed to be a function of the model itself. It is believed that in the chronic model, the degree of AHR decreases over time and, accordingly, becomes less responsive to treatment. In the art, the acute model is generally used to observe compound effects on airways responsiveness, and the chronic model is generally used to observe compound effects on airways remodelling. Remodelling assessments are ongoing. Nevertheless, it was surprising to observe the antibodies having an impact on the response to allergen-although the antibody didn't substantially impact absolute AHR (response to histamine) at this stage, it had significantly decreased the direct allergic response to antigen.

### 3.0.2. Inflammatory Bowel Disease

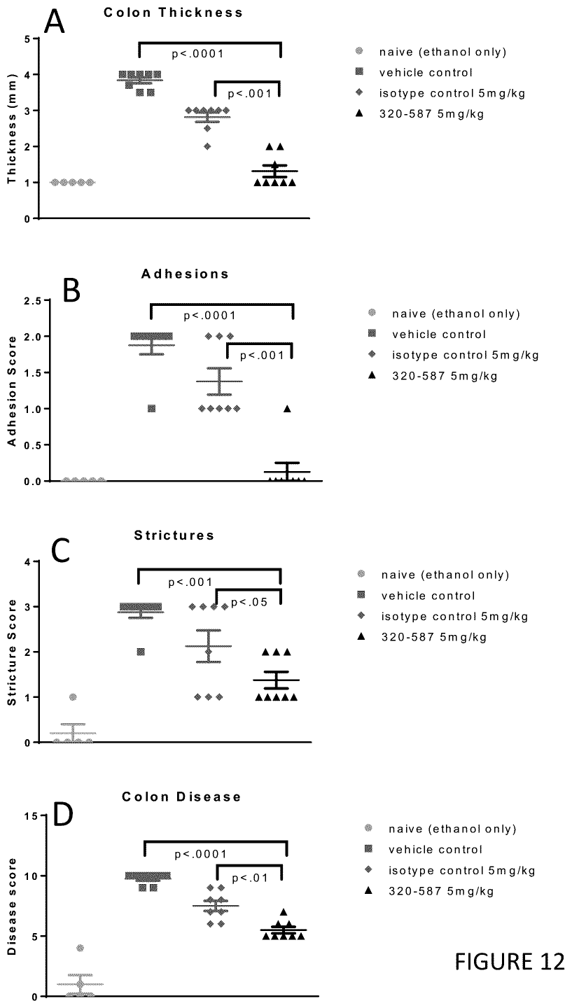

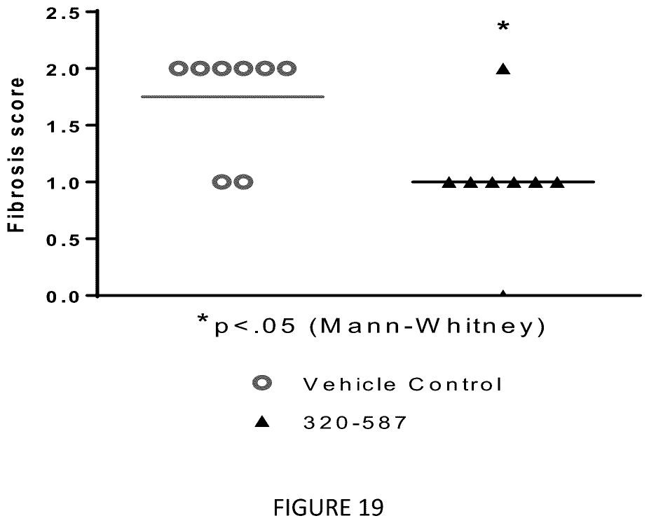

TNBS-induced colitis in rats: Rats were treated with a single dose of tri-nitrobenzenesulfonic acid in ethanol by intrarectal instillation dose. Control animals received equivalent volume of ethanol only. Over a space of 7 days, animals developed focal colitis characterized by ulceration of the colon with inflammatory infiltrate and varying degrees of fibrosis (e.g., Wirtz et al. (2007) Nat. Protoc. 2:541-546). 320-587 administration significantly reduced multiple disease indicators including colon thickness (FIG. 12A), number and severity of adhesions (FIG. 12B), and number and severity of strictures (FIG. 12C) leading to a significantly milder disease than animals treated with either vehicle or an isotype-matched irrelevant antibody (FIG. 12D). Decreased colon fibrosis (FIG. 19) was also observed in 320-587 treated animals.

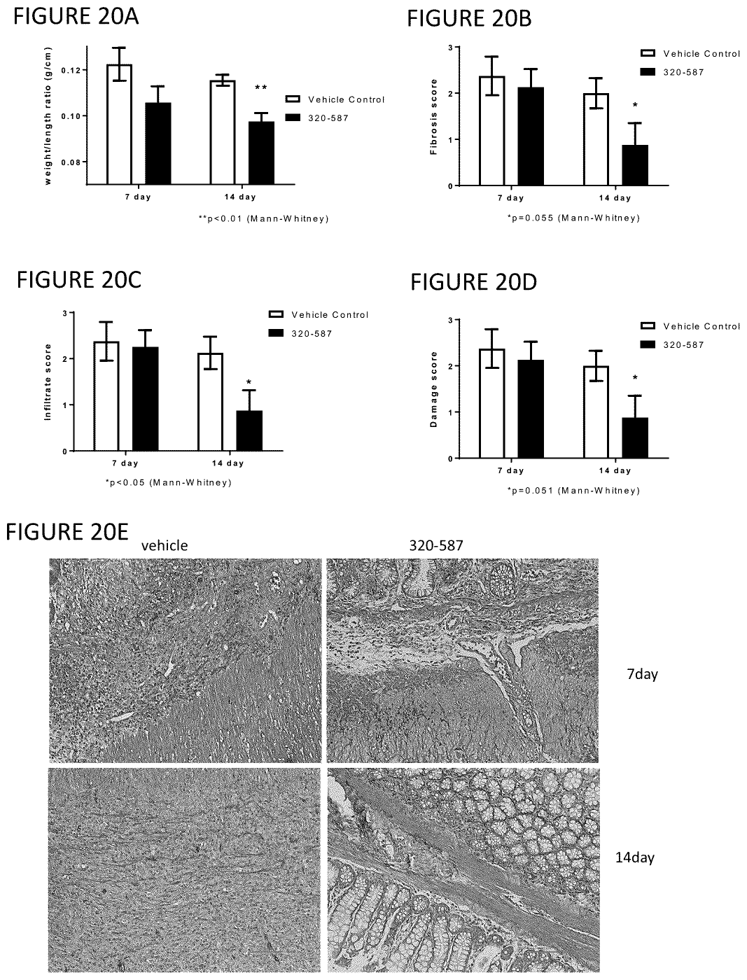

Comparison of disease after 7 and 14 days in DNBS-induced colitis in rats. Colitis was induced as described above using dinitrobenzenesulfonic acid (DNBS) instead of TNBS, and the rats used in the DNBS experiments were Wistar rats. Animals were treated with antibody 320-587 or vehicle i.v. on days 1 and 8. Groups were assessed for colitis 7 and 14 days post-DNBS and disease severity compared between the two timepoints. Treatment with antibody 320-587 had limited effect on day 7, but by day 14, animals treated with antibody 320-587 showed significant improvement in colon weight and length (FIG. 20A), fibrosis (FIG. 20B), inflammatory infiltrate (FIG. 20C) and colon damage (FIG. 20D). Representative sections of ulcer area colon (FIG. 20E) show the extent of damage repair and reduction in fibrosis at 14 days. At both 7 and 14 days, vehicle-treated animals showed extensive inflammatory infiltrate and fibrosis with significant loss of intestinal architecture. In contrast, 320-587-treated animals showed significant inflammatory infiltrate, fibrosis and loss of intestinal architecture at 7 days but these effects are largely reversed by 14 days.

The differences observed in the antibody therapeutic effects observed in the TNBS and DNBS models are believed to arise from the use of different strains of rats (Sprague-Dawley for TNBS versus Wistar for DNBS). Each rat strain has differences in their responses to immunological challenges such that the kinetics of their response in these models is believed to be different. As well, TNBS and DNBS are different structurally, and are believed to induce variations in the disease state.

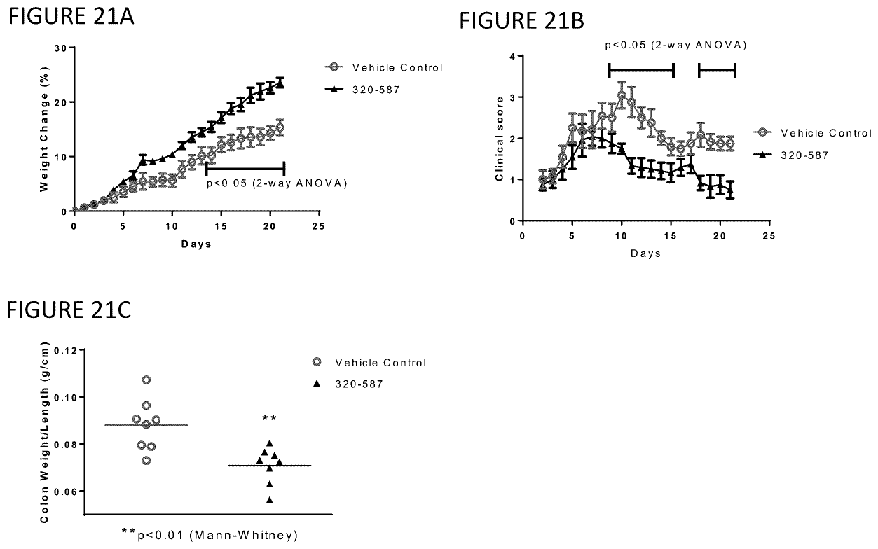

Chronic (21 day) DSS-induced colitis in rats. Rats were given dextran sulfate sodium (DSS) at a concentration of 5% w/v in drinking water for 7 days, then 2% w/v in drinking water for a further 14 days. Animals developed diarrhea, diffuse colonic inflammation, goblet cell hyperplasia, and crypt epithelial damage and ulceration (e.g. Randhawa et al. (2014) Korean J. Physiol. Pharmacol. 18:279-88). Rats were treated with antibody 320-587 or vehicle by intravenous injection on days 5, 12 and 19. Animals were weighed and assessed for clinical disease (diarrhea and occult blood) daily, and colon weight and length were assessed on day 21. Antibody 320-587 treatment significantly reversed DSS-induced slowdown of weight gain (FIG. 21A), ameliorated clinical signs of disease (FIG. 21B) and improved colon weight and length (FIG. 21C).

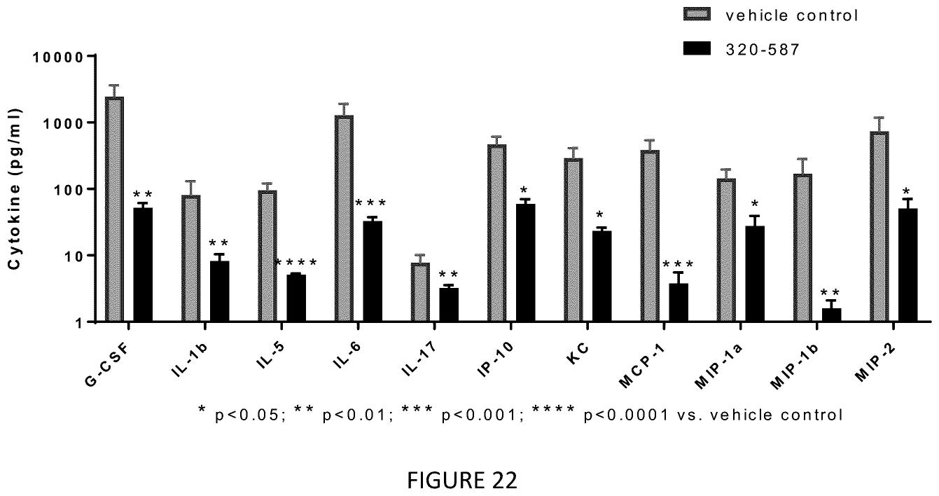

Induction of intraperitoneal cytokines by recombinant human TL1A. Intraperitoneal injection of recombinant mouse TL1A can induce the production of inflammatory cytokines such as IL-5. In this study, mice received a single dose of either antibody 320-587 or vehicle then an hour later were treated with recombinant human TL1A (rhTL1A) 40 μg/mouse. Six hours after rhTL1A dosage, peritoneal lavage was performed and the peritoneal fluid assessed for cytokines and chemokines by multiplex assay. Treatment with antibody 320-587 significantly decreased peritoneal concentrations of cytokines G-CSF, IL-1b, IL-5, IL-6, IL-17, and chemokines IP-10, KC, MCP-1, MIP-1a, MIP-1b, MIP-2 (FIG. 22).

The disclosure is not limited to the embodiments described and exemplified above, but is capable of variation and modification within the scope of the appended claims.

1-245. (canceled)

246. A method for treating a skin disease, comprising administering to a subject in need thereof a recombinant antibody that binds to TNF-like ligand 1A (TL1A), wherein the antibody comprises a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

247. The method according to claim 246, wherein the antibody comprises a human light chain lambda constant region.

248. The method according to claim 246, wherein the antibody comprises a human IgG1 heavy chain constant region and a human light chain lambda constant region.

249. The method according to claim 246, wherein the antibody comprises a human IgG1 heavy chain constant region and a human light chain lambda constant region.

250. The method according to claim 246, wherein the antibody comprises a heavy chain variable region comprising the amino acid sequence of SEQ ID NO:3.

251. The method according to claim 246, wherein the antibody comprises a light chain variable region comprising the amino acid sequence of SEQ ID NO:4.

252. The method according to claim 246, wherein the antibody comprises a heavy chain variable region comprising the amino acid sequence of SEQ ID NO:3 and a light chain variable region comprising the amino acid sequence of SEQ ID NO:4.

253. The method according to claim 246, wherein the antibody comprises a light chain comprising the amino acid sequence of SEQ ID NO:61.

254. The method according to claim 246, wherein the antibody comprises a heavy chain comprising the amino acid sequence of SEQ ID NO:60.

255. The method according to claim 246, wherein the antibody comprises a heavy chain comprising the amino acid sequence of SEQ ID NO:60 and a light chain comprising the amino acid sequence of SEQ ID NO:61.

256. The method according to claim 246, wherein the skin disease is atopic dermatitis, eczema, or scleroderma.

257. A method for treating atopic dermatitis, comprising administering to a subject in need thereof a recombinant human IgG1 antibody that binds to TNF-like ligand 1A (TL1A), wherein the antibody comprises a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

258. The method according to claim 257, wherein the antibody comprises a heavy chain variable region comprising the amino acid sequence of SEQ ID NO:3 and a light chain variable region comprising the amino acid sequence of SEQ ID NO:4.

259. The method according to claim 257, wherein the antibody comprises a heavy chain comprising the amino acid sequence of SEQ ID NO:60 and a light chain comprising the amino acid sequence of SEQ ID NO:61.

260. A method for treating eczema, comprising administering to a subject in need thereof a recombinant human IgG1 antibody that binds to TNF-like ligand 1A (TL1A), wherein the antibody comprises a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

261. The method according to claim 260, wherein the antibody comprises a heavy chain variable region comprising the amino acid sequence of SEQ ID NO:3 and a light chain variable region comprising the amino acid sequence of SEQ ID NO:4.

262. The method according to claim 260, wherein the antibody comprises a heavy chain comprising the amino acid sequence of SEQ ID NO:60 and a light chain comprising the amino acid sequence of SEQ ID NO:61.

263. A method for treating scleroderma, comprising administering to a subject in need thereof a recombinant human IgG1 antibody that binds to TNF-like ligand 1A (TL1A), wherein the antibody comprises a heavy chain variable region CDR1 comprising the amino acid sequence of SEQ ID NO:15, a heavy chain variable region CDR2 comprising the amino acid sequence of SEQ ID NO:21, a heavy chain variable region CDR3 comprising the amino acid sequence of SEQ ID NO:17, a light chain variable region CDR1 comprising the amino acid sequence of SEQ ID NO:18, a light chain variable region CDR2 comprising the amino acid sequence of SEQ ID NO:19, and a light chain variable region CDR3 comprising the amino acid sequence of SEQ ID NO:22.

264. The method according to claim 263, wherein the antibody comprises a heavy chain variable region comprising the amino acid sequence of SEQ ID NO:3 and a light chain variable region comprising the amino acid sequence of SEQ ID NO:4.

265. The method according to claim 263, wherein the antibody comprises a heavy chain comprising the amino acid sequence of SEQ ID NO:60 and a light chain comprising the amino acid sequence of SEQ ID NO:61.


SEQUENCE LISTING (104 sequences)

SEQ ID NO: 1 - protein, 119 aa, Homo sapiens
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 2 - protein, 111 aa, Homo sapiens
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 3 - protein, 119 aa, Homo sapiens
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 4 - protein, 111 aa, Homo sapiens
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 5 - protein, 111 aa, Homo sapiens
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 6 - protein, 111 aa, Homo sapiens
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 7 - protein, 111 aa, Homo sapiens
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 8 - protein, 111 aa, Homo sapiens
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 9 - protein, 111 aa, Homo sapiens
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSYDGTLSALFGGGTKLTVLG

SEQ ID NO: 10 - protein, 111 aa, Homo sapiens
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 11 - protein, 111 aa, Homo sapiens
QSVLTQPPSVSGAPGQRVTISCTSSSSDIGAALGVHWYQQLPGTAPKLLIEGYYNRPSGVPDRFSGSKSGTSASLTITGLLPEDEGDYYCQSYDGTLSALFGGGTKLTVLG

SEQ ID NO: 12 - protein, 111 aa, Homo sapiens
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSYDGTLSALFGGGTKLTVLG

SEQ ID NO: 13 - protein, 111 aa, Homo sapiens
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSYDGTLSALFGGGTKLTVLG

SEQ ID NO: 14 - protein, 111 aa, Homo sapiens
  VARIANT 32: note = G or A
  VARIANT 33: note = L or S or Q
  VARIANT 36: note = H or L
  VARIANT 93: note = Y or F or W
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 15 - protein, 10 aa, Homo sapiens
GYTFTSYDIN

SEQ ID NO: 16 - protein, 10 aa, Homo sapiens
WLNPNSGNTG

SEQ ID NO: 17 - protein, 10 aa, Homo sapiens
EVPETAAFEY

SEQ ID NO: 18 - protein, 14 aa, Homo sapiens
TSSSSDIGAGLGVH

SEQ ID NO: 19 - protein, 7 aa, Homo sapiens
GYYNRPS

SEQ ID NO: 20 - protein, 10 aa, Homo sapiens
QSYDGTLSAL

SEQ ID NO: 21 - protein, 10 aa, Homo sapiens
WLNPNSGYTG

SEQ ID NO: 22 - protein, 10 aa, Homo sapiens
QSWDGTLSAL

SEQ ID NO: 23 - protein, 14 aa, Homo sapiens
TSSSSDIGAALGVH

SEQ ID NO: 24 - protein, 14 aa, Homo sapiens
TSSSSDIGAGSGVH

SEQ ID NO: 25 - protein, 14 aa, Homo sapiens
TSSSSDIGAGQGVH

SEQ ID NO: 26 - protein, 14 aa, Homo sapiens
TSSSSDIGAGLGVL

SEQ ID NO: 27 - protein, 10 aa, Homo sapiens
QSFDGTLSAL

SEQ ID NO: 28 - protein, 10 aa, Homo sapiens
  VARIANT 8: note = N or Y
WLNPNSGXTG

SEQ ID NO: 29 - protein, 14 aa, Homo sapiens
  VARIANT 10: note = G or A
  VARIANT 11: note = L or S or Q
  VARIANT 14: note = H or L
TSSSSDIGAXXGVX

SEQ ID NO: 30 - protein, 10 aa, Homo sapiens
  VARIANT 3: note = Y or W or F
QSXDGTLSAL

SEQ ID NO: 31 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 32 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFAGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 33 - protein, 210 aa, Homo sapiens
DYKDDDDKGSHHHHHHHHGSGSLVPRGSGSLKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 34 - protein, 180 aa, Macaca fascicularis
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 35 - protein, 180 aa, Mus musculus
LRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 36 - protein, 180 aa, Rattus norvegicus
FPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 37 - protein, 179 aa, Cavia porcellus
INEQRFGPSYQRVYTPLRDDRDKPRAHLTVVRQTPTQHLKNQFPALHWEHELGLAFTKNRMNYTNKFLVIPETGDYFVYSQITFRGTTSECGISPGRQQNKPDSIFVVITKVTDSYPEPSQLLTGTKSVCEISSNWFQPLYLGAMFSLQEGDKLMVNVSDISLVDYTKEDKTFFGAFLL

SEQ ID NO: 38 - protein, 180 aa, Felis catus
PKGREFGPSHQRAYTSPGAGGDKPRAHLTVVRQTPTQPLKNQFPALHWEHELGLAFIKNRMNYTNKFLVIPESGDYFVYSQVTFRGTTSECGEIRQGSRLNKPDSIIVVITKVTDSYPEPTQLLMGTKSVCEVGSNWFQPIYLGAMFSLQEGDKLMVNVSDISLVDYTKEDKTFFGAFLL

SEQ ID NO: 39 - protein, 180 aa, Sus scrofa
PKGQELGPSHQRVYAPPGAGRDKPRAHLTVVRQTSTEPLKNQFPALHWEHELGLAFTKNRMNYTNKFLVIPESGDYFIYSQVTFRGTTSECGEISQERRLNKPDSIIVVITKVTDSYPEPTQLLMGTKSVCEIGSNWFQPIYLGAMFSLHEGDKLMVNVSDISLVDYTKEDKTFFGAFLL

SEQ ID NO: 40 - protein, 179 aa, Oryctolagus cuniculus
LKGREFGPSQQRAYMPLRADGNKPRAHLTAVKQTPTQPLRNHFPALHWEHELGLAFTKNRMNYTNKFLVIPESGDYFVYSQVTFRGTTSECGVINQRRRQTKPDSIVVVITKVTDNYPEPAQLLTGTKSVCEMGNWFQPIYLGAMFSLEEGDKLMVNVSDVSLVDYTKEDKTFFGAFLL

SEQ ID NO: 41 - protein, 180 aa, Canis lupus
PKGQEFGHSHQRAYASPRAGGDKPRAHLTVVRQSPTQPLESLFPALHWEHELGLAFTKNRMNYTNKFLVIPESGDYFVYSQVTFRGTTSECGEARQGSRLNKPDSIIVVITKVTDSYPEPTQLLMGTKSVCEVGSNWFQPIYLGAMFSLQEGDKLMVNVSDISLVDYTKEDKTFFGAFLL

SEQ ID NO: 42 - protein, 330 aa, Homo sapiens [sequence omitted]

SEQ ID NO: 43 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - DNA, 357 bp, Homo sapiens
  source 1..357: mol_type = genomic DNA
caggtgcagctggtgcagtctggggcggaggtgaagaagcctggggcctcagtgaaggtctcctgcaaggcttctggatacaccttcaccagttatgatatcaactgggtgcgacaggcccccggacaagggcttgagtggatgggatggctgaaccctaacagtggtaacacaggctatgcacagaagttccagggcagagtcaccatgaccgcagatcgttccaccagcacagcctacatggagctgagcagcctgagatctgaggacacggccgtgtattactgtgcgagagaagtgcctgagacggccgcctttgagtactggggccagggaaccctggtcaccgtctcctca

SEQ ID NO: 50 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctacgacggcaccctgagcgccctgttcggcggaggcaccaagctgaccgtcctaggt

SEQ ID NO: 51 - DNA, 357 bp, Homo sapiens
  source 1..357: mol_type = genomic DNA
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagc

SEQ ID NO: 52 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 53 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccgctctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 54 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggcagcggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 55 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggccagggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 56 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggcctgggcgtgctgtggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 57 - DNA, 333 bp, Homo sapiens
  source 1..333: mol_type = genomic DNA
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctttgacggcaccctgagcgccctgttcggcggaggcaccaagctgaccgtcctaggt

SEQ ID NO: 58 - DNA, 1344 bp, Homo sapiens
  source 1..1344: mol_type = genomic DNA
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagcgcctccaccaagggccccagcgtgttccccctggcccccagctccaagtccaccagcggcggaaccgccgctctgggctgcctggtgaaggactacttccccgagcccgtgaccgtgagctggaactccggcgccctgacctccggcgtgcacaccttccccgccgtgctgcagtccagcggcctgtactccctgagctccgtggtcaccgtgccctcctctagcctgggcacccagacctacatctgcaacgtgaaccacaagccctccaacaccaaggtggacaaaaaggtggagcccaagtcctgcgacaagactcacacctgtcctccctgccccgcccccgagctgctcggcggaccctccgtgttcctgttcccacccaagcccaaggacaccctgatgatctccaggacccccgaggtgacctgcgtggtcgtggacgtgtcccacgaggaccctgaggtgaagttcaactggtacgtggacggcgtggaggtgcacaacgccaagaccaagcccagggaggaacagtacaactccacctaccgggtcgtgtccgtgctgaccgtcctgcaccaggactggctgaacggcaaggagtacaagtgcaaggtgtccaacaaggccctgcccgcccccatcgagaagaccatctccaaggccaagggccagcctcgggagccccaggtgtacacactgcccccttccagggacgagctgaccaagaaccaggtgtccctgacctgtctggtgaagggcttctacccctccgacatcgccgtggagtgggagtccaacggccagcccgagaacaattacaagaccacacctcccgtcctggactccgacggctccttctttctgtactccaagctgaccgtggacaagtccaggtggcagcaaggcaacgtgttctcctgctccgtgatgcacgaggccctgcacaaccactacacccagaagtccctgagcctgtcccccggc

SEQ ID NO: 59 - DNA, 648 bp, Homo sapiens
  source 1..648: mol_type = genomic DNA
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggtcagcccaaggccgctcccagcgtgaccctgttccccccaagcagcgaggaactgcaggccaacaaggccaccctggtgtgcctgatcagcgacttctaccctggggccgtgaccgtggcctggaaggccgatagcagccctgtgaaggccggcgtggaaaccaccaccccctccaagcagagcaacaacaaatacgccgccagcagctacctgtccctgacccccgagcagtggaagtcccaccggtcctacagctgccaggtgacacacgagggcagcaccgtggaaaagaccgtggcccccaccgagtgcagc

SEQ ID NO: 60 - protein, 448 aa, Homo sapiens
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 216 aa, Homo sapiens
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 62 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 68 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 69 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 71 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 72 - protein, 10 aa, Homo sapiens
AAAAAAAAAA

SEQ ID NO: 73 - protein, 10 aa, Homo sapiens
SSSSSQSSSS

SEQ ID NO: 74 - protein, 10 aa, Homo sapiens
QQQQQDQQQQ

SEQ ID NO: 75 - protein, 10 aa, Homo sapiens
DDDDDHDHDD

SEQ ID NO: 76 - protein, 10 aa, Homo sapiens
HHHHHKHKHH

SEQ ID NO: 77 - protein, 10 aa, Homo sapiens
KKKKKLKLKK

SEQ ID NO: 78 - protein, 10 aa, Homo sapiens
LLLLLWLWLL

SEQ ID NO: 79 - protein, 10 aa, Homo sapiens
WWWWWYWYWW

SEQ ID NO: 80 - protein, 12 aa, Homo sapiens
AAAAAAAAAAAS

SEQ ID NO: 81 - protein, 12 aa, Homo sapiens
SSSSSQSSSSSQ

SEQ ID NO: 82 - protein, 12 aa, Homo sapiens
QQQQQDQQQQQD

SEQ ID NO: 83 - protein, 12 aa, Homo sapiens
DDDDDHDDDDDH

SEQ ID NO: 84 - protein, 12 aa, Homo sapiens
HHHHHKHHHHKH

SEQ ID NO: 85 - protein, 12 aa, Homo sapiens
KKKKKLKKKKKL

SEQ ID NO: 86 - protein, 12 aa, Homo sapiens
LWLLLWLLLLLW

SEQ ID NO: 87 - protein, 12 aa, Homo sapiens
YYWWWYWWWWWY

SEQ ID NO: 88 - protein, 7 aa, Homo sapiens
YYYYYYY

SEQ ID NO: 89 - protein, 14 aa, Homo sapiens
AAAAAAAASAAAAA

SEQ ID NO: 90 - protein, 14 aa, Homo sapiens
SQQQQSSSQSSSSS

SEQ ID NO: 91 - protein, 14 aa, Homo sapiens
QDDDDQQQDQQQQQ

SEQ ID NO: 92 - protein, 14 aa, Homo sapiens
DHHHHHDDHDDDDD

SEQ ID NO: 93 - protein, 14 aa, Homo sapiens
HKKKKKHHKHHHHH

SEQ ID NO: 94 - protein, 14 aa, Homo sapiens
KLLLLLKKLKKKKK

SEQ ID NO: 95 - protein, 14 aa, Homo sapiens
LWWWWWLLWLWLLL

SEQ ID NO: 96 - protein, 14 aa, Homo sapiens
WYYYYYWWYWYWWW

SEQ ID NO: 97 - protein, 10 aa, Homo sapiens
AAAAAAAASA

SEQ ID NO: 98 - protein, 10 aa, Homo sapiens
SQSSSSSQQS

SEQ ID NO: 99 - protein, 10 aa, Homo sapiens
DDQQQQQDDQ

SEQ ID NO: 100 - protein, 10 aa, Homo sapiens
HHDHDDDHHD

SEQ ID NO: 101 - protein, 10 aa, Homo sapiens
KKHKHHHKKH

SEQ ID NO: 102 - protein, 10 aa, Homo sapiens
LLKLKKKLLK

SEQ ID NO: 103 - protein, 10 aa, Homo sapiens
WWLWLLWWWW

SEQ ID NO: 104 - protein, 10 aa, Homo sapiens
YYWYWWYYYY